SEQUENCE LISTING (24 sequences)

SEQ ID NO: 1 - protein, 5 aa, Artificial Sequence
  Anti-PD1 ISVD  CDR 1
IHAMG

SEQ ID NO: 2 - protein, 17 aa, Artificial Sequence
  Anti-PD1 ISVD  CDR 2 (variant 1)
VITWSGGITYYADSVKG

SEQ ID NO: 3 - protein, 17 aa, Artificial Sequence
  Anti-PD1 ISVD  CDR 2 (variant 2)
VITVSGGITYYADSVKG

SEQ ID NO: 4 - protein, 10 aa, Artificial Sequence
  Anti-PD1 ISVD  CDR 3 (variant 1)
DKHQSSWYDY

SEQ ID NO: 5 - protein, 10 aa, Artificial Sequence
  Anti-PD1 ISVD  CDR 3 (variant 2)
DKHQSSFYDY

SEQ ID NO: 6 - protein, 5 aa, Artificial Sequence
  Anti-LAG3 ISVD  CDR 1
DYVMG

SEQ ID NO: 7 - protein, 10 aa, Artificial Sequence
  Anti-LAG3 ISVD  CDR 2
AISESGGRTH

SEQ ID NO: 8 - protein, 19 aa, Artificial Sequence
  Anti-LAG3 ISVD  CDR 3
TLLWWTSEYAPIKANDYDY

SEQ ID NO: 9 - protein, 10 aa, Artificial Sequence
  Anti-CTLA4 ISVD  CDR 1
GGTFSFYGMG

SEQ ID NO: 10 - protein, 10 aa, Artificial Sequence
  Anti-CTLA4 ISVD  CDR 2
DIRTSAGRTY

SEQ ID NO: 11 - protein, 10 aa, Artificial Sequence
  Anti-CTLA4 ISVD  CDR 3 (variant 1)
EMSGISGWDY

SEQ ID NO: 12 - protein, 10 aa, Artificial Sequence
  Anti-CTLA4 ISVD  CDR 3 (variant 2)
EPSGISGWDY

SEQ ID NO: 13 - protein, 5 aa, Artificial Sequence
  Anti-HSA ISVD  CDR 1
SFGMS

SEQ ID NO: 14 - protein, 10 aa, Artificial Sequence
  Anti-HSA ISVD  CDR 2
SISGSGSDTL

SEQ ID NO: 15 - protein, 6 aa, Artificial Sequence
  Anti-HSA ISVD  CDR 3
GGSLSR

SEQ ID NO: 16 - protein, 119 aa, Artificial Sequence
  Anti-PD1 VHH  (ISVD )
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 17 - protein, 119 aa, Artificial Sequence
  Anti-PD1 VHH  (ISVD ) D1E variant
EVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 18 - protein, 128 aa, Artificial Sequence
  Anti-LAG3 VHH (ISVD )
EVQLVESGGGVVQPGGSLRLSCAASGRTFSDYVMGWFRQAPGKEREFVAAISESGGRTHYADSVKGRFTISRDNSKNTLYLQMNSLRPEDTALYYCATTLLWWTSEYAPIKANDYDYWGQGTLVTVSS

SEQ ID NO: 19 - protein, 128 aa, Artificial Sequence
  Anti-LAG3 VHH (ISVD ) E1D variant
DVQLVESGGGVVQPGGSLRLSCAASGRTFSDYVMGWFRQAPGKEREFVAAISESGGRTHYADSVKGRFTISRDNSKNTLYLQMNSLRPEDTALYYCATTLLWWTSEYAPIKANDYDYWGQGTLVTVSS

SEQ ID NO: 20 - protein, 115 aa, Artificial Sequence
  Anti-HSA VHH (ISVD )
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 21 - protein, 115 aa, Artificial Sequence
  Anti-HSA VHH (ISVD ) E1D variant
DVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 22 - protein, 119 aa, Artificial Sequence
  Anti-CTLA4 VHH (ISVD )
DVQLVESGGGVVQPGGSLRLSCAASGGTFSFYGMGWFRQAPGKEREFVADIRTSAGRTYYADSVKGRFTISRDNSKNTVYLQMNSLRPEDTALYYCAAEPSGISGWDYWGQGTLVTVSS

SEQ ID NO: 23 - protein, 119 aa, Artificial Sequence
  Anti-CTLA4 VHH (ISVD)  D1E variant
EVQLVESGGGVVQPGGSLRLSCAASGGTFSFYGMGWFRQAPGKEREFVADIRTSAGRTYYADSVKGRFTISRDNSKNTVYLQMNSLRPEDTALYYCAAEPSGISGWDYWGQGTLVTVSS

SEQ ID NO: 24 - protein, 433 aa, Artificial Sequence
  MSD-21 polypeptide (PD1 )-(LAG3 )-(HSA )
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSGGGGSGGGGSGGGGSGGGGSGGGGSGGGGSGGGGSEVQLVESGGGVVQPGGSLRLSCAASGRTFSDYVMGWFRQAPGKEREFVAAISESGGRTHYADSVKGRFTISRDNSKNTLYLQMNSLRPEDTALYYCATTLLWWTSEYAPIKANDYDYWGQGTLVTVSSGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

(19) United States
(12) Patent Application Publication
(10) Pub. No.: US 2022/0267370 A1
(43) Pub. Date: Aug. 25, 2022

(54) Process for Separating Antigen-Binding Polypeptide Monomers Comprising One or More Immunoglobulin Single Variable Domains from Aggregates of Said Monomers

(71) Applicants: Ehsan Allah ESPAH BORUJENI, Kenilworth, NJ (US); William J. RAYFIELD, Kenilworth, NJ (US); Sandra E. RIOS, Kenilworth, NJ (US); Merck Sharp & Dohme Corp., Rahway, NJ (US)

(72) Inventors: Ehsan Allah Espah Borujeni, Jersey City, NJ (US); William J. Rayfield, High Bridge, NJ (US); Sandra E. Rios, Berkeley, NJ (US)

(73) Assignee: Merck Sharp & Dohme Corp., Rahway, NJ (US)

(21) Appl. No.: 17/617,782

(22) PCT Filed: Jun. 22, 2020
(86) PCT No.: PCT/US2020/038870

§ 371 Date: Dec. 9, 2021

Related U.S. Application Data
(60) Provisional application No. 62/866,923, filed on Jun. 26, 2019.

(51) Int. Cl. C07K 1/22; C07K 1/36

(52) U.S. Cl. CPC C07K 1/22; C07K 1/36

(57) ABSTRACT

A method that uses Protein A chromatography to separate antigen-binding polypeptide monomers comprising one or more immunoglobulin single variable domains (ISVDs) from aggregates of said monomers is disclosed.

### BACKGROUND OF THE INVENTION

### (1) Field of the Invention

The present invention provides a process that uses Protein A chromatography to separate antigen-binding polypeptide monomers comprising one or more immunoglobulin single variable domains (ISVDs) from aggregates or high molecular weight species of said monomers.

### (2) Description of Related Art

Over the past 15 to 20 years, monoclonal antibodies (mAbs) have become a successful class of therapeutic products. However, the manufacture of safe and effective mAb drug products presents many challenges to downstream purification. Removal of high molecular weight species or aggregates, especially soluble aggregates, presents a challenge due to the physical and chemical similarity of the aggregates to the drug product itself. Chromatography steps can effectively remove aggregates, and, typically, one or more chromatography steps in a bioprocess will be optimized for aggregate removal. The need for aggregate removal, however, must be balanced by the productivity of the bioprocess, the step yield, and the overall purity of the product through the removal of host-cell proteins and other contaminants.

With respect to manufacturing monoclonal antibody products, nearly all bioprocesses begin with an initial Protein A affinity chromatography step to remove the bulk of impurities present in the clarified harvest from cell culture. This initial step provides a product that is typically greater than 90/o pure, and the subsequent bioprocessing steps are focused on the removal of the remaining minor impurities. Consequently, by introducing the Protein A step early in the overall bioprocess for manufacturing product, the number of successive unit operations can be reduced. Protein A chromatography is fast and easy to use: methods for purifying antibodies on protein A are well known in the art. See for example, U.S. Pat. Nos. 8,895,709; 9,018,361; 9,556,258, and U.S. Pat. Pubs. 20110144311 and 20130178608.

While Protein A chromatography can effectively remove a significant amount of contaminants from the antibody preparation, it does not effectively remove aggregates already present in the feedstock because of the chemical similarity of aggregates to the single antibody molecule. To prevent further aggregation due to denaturation at the acidic pHs typically used in Protein A chromatography, the Protein A elution conditions must be optimized. Finally, Protein A eluates are usually maintained at a low pH for 30-60 minutes as a viral inactivation measure. This hold has the potential to exacerbate aggregate formation.

Downstream purification steps are often used to remove aggregates following Protein A chromatography and low pH treatment. Because aggregates are multiples of non-aggregated antibody molecules, aggregates will have proportionately greater surface charge or surface hydrophobicity. Ion (anion or cation) exchange and hydrophobic interaction chromatography may be used to take advantage of this increased charge and hydrophobicity of the aggregates to separate them from the non-aggregated antibody molecules.

Certain antibody fragments such as Fabs and F(ab′)2 and immunoglobulin single variable domains (ISVDs), which are antibody fragments comprising a single monomeric variable antibody domain (for example, V_{HH} fragments derived from Camelid heavy chain-only antibodies and scFvs derived from standard antibodies), have been shown to bind Protein A. See for example, Sasso et al. J. Immunol. 147: 1877-1883 (1991); Frenken et al., J. Biotechnol. 78: 11-21 (2000); Fridy et al., Nat. Meth. 11: 1253-1260 (2014); Fridy et al., Anal. Biochem. 477: 92-94, (2015); Intl. Patent Appl. No. WO2009068627; and, U.S. Pat. No. 10,118,962. However, since ISVDs do not have an Fc domain, the interaction of the ISVD with Protein A involves various residues in the variable domain. Fridy et al. op. cit. (2015), has shown that V_{HH} fragments have two amino acids in the variable domain that appear to be required for binding to Protein A. While antibody fragments and ISVDs have been shown to bind Protein A, antibody fragments and ISVDs lack an Fc domain, thus their binding is not similar to that of an antibody. This differential binding suggests that purifying antibody fragments and ISVDs may require modifications to the Protein A processes that have been used for purifying antibodies.

### BRIEF SUMMARY OF THE INVENTION

The present invention provides a process for isolating antigen-binding polypeptide (ABP) monomers comprising one or more immunoglobulin single variable domains (ISVDs) from high molecular weight species (or aggregates) of the ABP monomers that may be present in a harvested cell culture fluid (HCCF) obtained from cell cultures comprising cells expressing the ABP monomers. The method comprises applying the HCCF to a column comprising a Protein A resin selected from the group consisting of TOYOPEARL AF-rProtein A HC-650F resin or AMSPHERE A3 Protein A resin, each equilibrated in an equilibration solution at a neutral pH; washing the column with a wash solution at a neutral pH; and eluting the ABP monomers from the column with an elution solution at a pH of about 3.5 to obtain an eluent comprising the ABP monomers separated from aggregates of the ABP monomers.

The inventors have discovered that Protein A resins from different suppliers bind aggregates of ABP monomers to different extents or avidity such that for certain resins under typical Protein A elution conditions (i.e., pH 3.0 or less), the aggregates co-elute with the ABP monomers, but for the TOYOPEARL AF-rProtein A HC-650F or AMSPHERE A3 Protein A resins, aggregates of ABP monomers are bound more strongly or with greater avidity than ABP monomers under conditions performed at pH 3.5. Thus, during elution of the ABP monomers from the TOYOPEARL AF-rProtein A HC-650F or AMSPHERE A3 Protein A resins at pH 3.5, more of the aggregates will remain bound to either resin. Thus, the discovery has led to the present invention in which Protein A column chromatography is used to separate ABP monomers from aggregates of such monomers.

Therefore, while the HCCF comprises ABP monomers in a mixture with aggregates of the ABP monomers and other contaminants from the cell culture fermentation process, the Protein A chromatography method of the present invention enables the separation of the ABP monomers from aggregates of the ABP monomers to provide a composition that is substantially free of aggregates of the ABP monomers. In particular embodiments, the resulting composition comprises ABP monomers and either (i) aggregates of the ABP monomers that are less than about 2% as may be obtained when the protein load applied to the Protein A chromatography column is about 20 grams protein/liter resin or less or (ii) less than about 1.6% when the protein load applied to the Protein A chromatography column is greater than about 20 grams protein/liter resin or about 40 grams protein/liter resin.

The present invention provides a process for separating antigen-binding polypeptide (ABP) monomers from aggregates of the ABP monomers in a harvested cell culture fluid (HCCF), comprising (a) providing a HCCF from a culture of recombinant cells expressing ABP monomers, wherein the total protein in the HCCF comprises a mixture of the ABP monomers, aggregates of the ABP monomers, and other protein, and a chromatography column comprising TOYOPEARL AF-rProtein A HC-650F resin or AMSPHERE A3 resin equilibrated in an equilibration solution at a slightly acidic pH or neutral pH; (b) applying the HCCF to the chromatography column; (c) washing the chromatography column with at least one wash solution at a neutral pH or slightly acidic pH; and (d) eluting the polypeptide monomers from the chromatography column with an elution solution at about pH 3.5 to obtain an eluent comprising the ABP monomer, wherein the eluant comprises less than about 5% of the aggregates of the ABP monomers as determined by ultra-performance size exclusion chromatography and wherein an ABP monomer comprises one or more immunoglobulin single variable domains (ISVDs). In particular embodiments, the eluant comprises less than about 2% of the aggregates of the ABP monomers and in further embodiments, the eluant comprises less than about 1.6% of the aggregates of the ABP monomers.

As used in the present invention, a slightly acidic pH is a pH that is greater than about pH 6.0 and less than pH 7.0 and a neutral pH is a pH of 7.0 to about 7.5. In particular embodiments, slightly acidic pH is about pH 6.5.

In a further embodiment of the process, the HCCF is applied to the column at a continuous flow rate until the amount of total protein applied to the column reaches an amount to be about 10% breakthrough or the amount of total protein applied to the column is about 9 to 18 grams protein/liter resin. In particular aspects, the total protein concentration of the HCCF comprises about 1.0 grams/liter to about 1.5 grams/liter of protein. In particular aspects, the flow rate may be up to about 300 cm/hr. The 10% breakthrough may be predetermined in an assay to determine the amount of total protein that can be applied to the column that begins to flow through the column to determine the breakthrough amount and then applying to the column HCCF until the amount of protein loaded on the column is about 10% of the breakthrough amount.

In a further embodiment of the process, the equilibration solution and the at least one wash solution comprises sodium phosphate. In particular embodiments, the equilibration solution and the at least one wash solution each comprises about 10 to 20 mM sodium phosphate. In particular embodiments, the equilibration solution and the at least one wash solution each has a pH of about pH 6.5. In particular embodiments, the column is washed with two wash solutions: a first wash solution at about pH 6.5 comprising of sodium phosphate and a second wash solution at about pH 6.5 comprising of sodium phosphate and sodium chloride. In a further embodiment, the first and second wash solutions comprise about 10 to 20 mM sodium phosphate and the second wash solution further comprises about 500 mM sodium chloride. In a further still embodiment, the first and second wash solutions comprise about 10 mM sodium phosphate and the second wash solution further comprises about 500 mM sodium chloride. In a further embodiment, the column is washed with the first wash solution, then washed with the second wash solution, and finally washed with the first wash solution. In particular embodiments, the column is washed with about three column volumes (CVs) of the first wash solution, then washed with about five CVs of the second wash solution, and finally washed with about five CVs of the first wash solution.

In a further embodiment of the process, the ABP monomers are eluted from the column with an elution solution comprising sodium acetate at about pH 3.5. In particular aspects, the elution solution comprises sodium acetate at a concentration of about 10 to about 100 mM. In further embodiments, the sodium acetate may be at a concentration of about 20 to 100 mM, or in particular embodiments at a concentration of about 20 mM or 100 mM.

In a further embodiment of the process, each of the ISVDs comprises a humanized Camelid variable heavy domain (V_{HH}), which in further embodiments comprise an arginine residue at position 19 and an asparagine residue at position 82a wherein the position numbers are according to Kabat.

In particular embodiments of the process, the ABP monomer comprises at least one ISVD that comprises an amino acid sequence that binds programmed death receptor 1 (PD-1). In a further embodiment, the ABP monomer comprises at least two ISVDs wherein one ISVD has an amino acid sequence that binds programmed death receptor 1 (PD-1) and the other ISVD or ISVDs have amino acid sequences that bind another antigens.

In a further embodiment of the process, the ABP monomers comprise (i) at least one ISVD amino acid sequence that binds PD-1 and at least one ISVD amino acid sequence that binds human serum albumin (HSA); (ii) at least one ISVD amino acid sequence that binds PD-1 and at least one ISVD amino acid sequence that binds lymphocyte activation gene 3 (LAG3); (iii) at least one ISVD amino acid sequence that binds PD-1, at least one ISVD amino acid sequence that binds LAG3, and at least one ISVD amino acid sequence that binds HSA; (iv) at least one ISVD amino acid sequence that binds PD-1 and at least one ISVD amino acid sequence that binds cytotoxic T-lymphocyte-associated protein 4 (CTLA4); or (v) at least one ISVD amino acid sequence that binds PD-1, at least one ISVD amino acid sequence that binds cytotoxic CTLA4, and at least one ISVD amino acid sequence that binds HSA.

In further embodiments of the process, the ISVD amino acid sequence that binds PD-1 is a camelid variable domain (V_{HH}) amino acid sequence comprising a complementarity determining region (CDR) 1 having the amino acid sequence set forth in SEQ ID NO: 1; a CDR 2 having the amino acid sequence set forth in SEQ ID NO: 2 or 3; and a CDR 3 having the amino acid sequence set forth in SEQ ID NO: 4 or 5.

In further embodiments of the process, the ISVD amino acid sequence that binds LAG3 is a camelid variable domain (V_{HH}) amino acid sequence comprising a complementarity determining region (CDR) 1 having the amino acid sequence set forth in SEQ ID NO: 6; a CDR 2 having the amino acid sequence set forth in SEQ ID NO: 7; and a CDR 3 having the amino acid sequence set forth in SEQ ID NO: 8.

In further embodiments of the process, the ISVD amino acid sequence that binds HSA is a camelid variable domain (V_{HH}) amino acid sequence comprising a complementarity determining region (CDR) 1 having the amino acid sequence set forth in SEQ ID NO: 13; a CDR 2 having the amino acid sequence set forth in SEQ ID NO: 14; and a CDR 3 having the amino acid sequence set forth in SEQ ID NO: 15.

In further embodiments of the process, the ISVD amino acid sequence that binds CTLA4 is a camelid variable domain (V_{HH}) amino acid sequence comprising a complementarity determining region (CDR) 1 having the amino acid sequence set forth in SEQ ID NO: 9; a CDR 2 having the amino acid sequence set forth in SEQ ID NO: 10; and a CDR 3 having the amino acid sequence set forth in SEQ ID NO: 11 or 12.

In further embodiments of the process, the ISVD amino acid sequence that binds PD-1 comprises the amino acid sequence set forth in SEQ ID NO: 16 or 17; the ISVD amino acid sequence that binds LAG3 comprises the amino acid sequence set forth in SEQ ID NO: 18 or 19; the ISVD amino acid sequence that binds HSA comprises the amino acid sequence set forth in SEQ ID NO: 20 or 21; and the ISVD amino acid sequence that binds CTLA4 comprises the amino acid sequence set forth in SEQ ID NO: 22 or 23.

In further embodiments of the process, the ABP monomer is multispecific polypeptide that binds PD-1 and LAG3 and comprises the amino acid sequence set forth in SEQ ID NO:24.

The present invention further provides a composition comprising antigen-binding polypeptide (ABP) monomers and a pharmaceutically acceptable carrier, wherein less than about 5% of the ABP monomers are in aggregates as determined by ultra-performance size exclusion chromatography and wherein the ABP monomers comprise one or more immunoglobulin single variable domains (ISVDs), wherein the composition is obtained from the Protein A chromatography process disclosed herein. In particular embodiments, the composition comprises less than about 2% of the aggregates of the ABP monomers and in further embodiments, the composition comprises less than about 1.6% of the aggregates of the ABP monomers.

In a further embodiment of the composition, each of the ISVDs comprises a humanized Camelid variable heavy domain (V_{HH}), which in further embodiments comprises an arginine residue at position 19 and an asparagine residue at position 82a wherein the position numbers are according to Kabat.

In particular embodiments of the composition, the ABP monomer comprises at least one ISVD that comprises an amino acid sequence that binds programmed death receptor 1 (PD-1). In a further embodiment, the ABP monomer comprises at least two ISVDs wherein one ISVD has an amino acid sequence that binds programmed death receptor 1 (PD-1) and the other ISVD or ISVDs have amino acid sequences that bind another antigens.

In a further embodiment of the composition, the ABP monomers comprise (i) at least one ISVD amino acid sequence that binds PD-1 and at least one ISVD amino acid sequence that binds human serum albumin (HSA); (ii) at least one ISVD amino acid sequence that binds PD-1 and at least one ISVD amino acid sequence that binds lymphocyte activation gene 3 (LAG3); (iii) at least one ISVD amino acid sequence that binds PD-1, at least one ISVD amino acid sequence that binds LAG3, and at least one ISVD amino acid sequence that binds HSA; (iv) at least one ISVD amino acid sequence that binds PD-1 and at least one ISVD amino acid sequence that binds cytotoxic T-lymphocyte-associated protein 4 (CTLA4); or (v) at least one ISVD amino acid sequence that binds PD-1, at least one ISVD amino acid sequence that binds cytotoxic CTLA4, and at least one ISVD amino acid sequence that binds HSA.

In further embodiments of the composition, the ISVD amino acid sequence that binds PD-1 is a camelid variable domain (V_{HH}) amino acid sequence comprising a complementarity determining region (CDR) 1 having the amino acid sequence set forth in SEQ ID NO: 1; a CDR 2 having the amino acid sequence set forth in SEQ ID NO: 2 or 3; and a CDR 3 having the amino acid sequence set forth in SEQ ID NO: 4 or 5.

In further embodiments of the composition, the ISVD amino acid sequence that binds LAG3 is a camelid variable domain (V_{HH}) amino acid sequence comprising a complementarity determining region (CDR) 1 having the amino acid sequence set forth in SEQ ID NO: 6; a CDR 2 having the amino acid sequence set forth in SEQ ID NO: 7; and a CDR 3 having the amino acid sequence set forth in SEQ ID NO: 8.

In further embodiments of the composition, the ISVD amino acid sequence that binds HSA is a camelid variable domain (V_{HH}) amino acid sequence comprising a complementarity determining region (CDR) 1 having the amino acid sequence set forth in SEQ ID NO: 13; a CDR 2 having the amino acid sequence set forth in SEQ ID NO: 14; and a CDR 3 having the amino acid sequence set forth in SEQ ID NO: 15.

In further embodiments of the composition, the ISVD amino acid sequence that binds CTLA4 is a camelid variable domain (V_{HH}) amino acid sequence comprising a complementarity determining region (CDR) 1 having the amino acid sequence set forth in SEQ ID NO: 9; a CDR 2 having the amino acid sequence set forth in SEQ ID NO: 10; and a CDR 3 having the amino acid sequence set forth in SEQ ID NO: 11 or 12.

In further embodiments of the composition, the ISVD amino acid sequence that binds PD-1 comprises the amino acid sequence set forth in SEQ ID NO: 16 or 17; the ISVD amino acid sequence that binds LAG3 comprises the amino acid sequence set forth in SEQ ID NO: 18 or 19; the ISVD amino acid sequence that binds HSA comprises the amino acid sequence set forth in SEQ ID NO: 20 or 21; and the ISVD amino acid sequence that binds CTLA4 comprises the amino acid sequence set forth in SEQ ID NO: 22 or 23.

In further embodiments of the composition, the ABP monomer is multispecific polypeptide that binds PD-1 and LAG3 and comprises the amino acid sequence set forth in SEQ ID NO:24.

### BRIEF DESCRIPTION OF THE DRAWINGS

FIG. 1: Results of a slurry plate affinity resin screening (Run 1) for ABP monomer MSD-21. See Table 6 for design of the experiment.

FIG. 2A: Results of a second slurry plate affinity resin screening (Run 2) for ABP monomer MSD-21. See Table 7 for design of the experiment.

FIG. 2B: UP-SEC chromatograms of the results shown in FIG. 2A. The upper panel shows the relative amounts of ABP monomer MSD-21 and aggregates of the MSD-21 monomer (HMW) in eluant obtained from MabSelect SuRe™ resin at pH 3.0 vs. pH 3.5. The lower panel shows the relative amounts of ABP monomer MSD-21 and aggregates of the MSD-21 monomer (HMW) in eluant obtained from TOYOPEARL AF-rProtein A HC-650F resin at pH 3.0 vs. pH 3.5. HMW brackets indicate area where HMW elute from column.

FIG. 2C: zoom views of the UP-SEC chromatograms in FIG. 2B. The upper panel shows the relative amounts of ABP monomer MSD-21 and aggregates of the MSD-21 monomer (HMW) in eluant obtained from MabSelect SuRe™ resin at pH 3.0 vs. pH 3.5. The lower panel shows the relative amounts of ABP monomer MSD-21 and aggregates of the MSD-21 monomer (HMW) in eluant obtained from TOYOPEARL AF-rProtein A HC-650F resin at pH 3.0 vs. pH 3.5. HMW brackets indicate area where HMW elute from column.

FIG. 2D: zoom views of the UP-SEC chromatograms shown in FIG. 2C wherein the chromatogram tracings for the MabSelect SuRe™ and TOYOPEARL AF-rProtein A HC-650F resin eluants for each pH are superimposed. The upper panel shows the relative amounts of ABP monomer MSD-21 and aggregates of the MSD-21 monomer (HMW) in eluants obtained from MabSelect SuRe™ and TOYOPEARL AF-rProtein A HC-650F resins at pH 3.0. The lower panel shows the relative amounts of ABP monomer MSD-21 and aggregates of the MSD-21 monomer (HMW) in eluants obtained from MabSelect SuRe™ and TOYOPEARL AF-rProtein A HC-650F resins at pH 3.5. HMW brackets indicate area where HMW elute from column.

FIG. 3: Dynamic binding concentration (DBC) profiles of four different Protein A affinity resins for binding MSD-21 monomer.

FIG. 4: UP-SEC chromatograms of MSD-21 monomer Protein A pool (PAP) fraction and MSD-21 monomer aggregate Protein A Strip (PAST) fraction (column stripped of bound aggregate following elution of the monomer). The PAP and PAST fraction chromatograms are superimposed to show the relative positions of the PAP and PAST fractions. HMW=aggregates of MSD-21. HMW brackets indicate area where HMW elute from column.

FIG. 5: UP-SEC chromatograms of the eluant from a series of MSD-21 Protein A Load Solutions spiked with different amounts of MSD-21 aggregates (HMW) obtained from the PAST fraction. The results further show the relative positions of the HMW aggregates in PAP fractions. HMW brackets indicate area where HMW elute from column.

FIG. 6A: HMW aggregate amount in MSD-21 Protein A product pools (PAPs) as a function of Protein A resin, load HMW aggregate amount added to feed, and feed protein/liter resin concentration. HMW=aggregates of MSD-21. The feed protein concentration at about the dynamic binding capacity (DBC) for the resin is about 12.1 grams protein/liter resin for MabSelect SuRe™ or 15.9 grams protein/liter resin for TOYOPEARL AF-rProtein A HC-650. HMW brackets indicate area where HMW elute from column.

FIG. 6B: UP-SEC chromatograms of the PAPs from the MabSelect SuRe™ and TOYOPEARL AF-rProtein A HC-650 resins, each loaded with a feed containing 3.6% HMW and either 12.1 grams protein/liter resin for MabSelect SuRe™ or 15.9 grams protein/liter resin for TOYOPEARL AF-rProtein A HC-650 shown in FIG. 6A. HMW=aggregates of MSD-21. HMW brackets indicate area where HMW elute from column.

FIG. 6C: UP-SEC chromatograms of the PAPs from the MabSelect SuRe™ and TOYOPEARL AF-rProtein A HC-650 resins, each loaded with a feed containing 7.4% HMW and either 12.1 grams protein/liter resin for MabSelect SuRe™ or 15.9 grams protein/liter resin for TOYOPEARL AF-rProtein A HC-650 shown in FIG. 6A. HMW=aggregates of MSD-21. HMW brackets indicate area where HMW elute from column.

FIG. 6D: UP-SEC chromatograms of the PAPs from the MabSelect SuRe™ and TOYOPEARL AF-rProtein A HC-650 resins, each loaded with a feed containing 10.2% HMW and either 12.1 grams protein/liter resin for MabSelect SuRe™ or 15.9 grams protein/liter resin for TOYOPEARL AF-rProtein A HC-650 shown in FIG. 6A. HMW=aggregates of MSD-21. HMW brackets indicate area where HMW elute from column.

FIG. 6E: UP-SEC chromatograms of the PAPs from the MabSelect SuRe™ and TOYOPEARL AF-rProtein A HC-650 resins, each loaded with a feed containing 25.4% HMW and either 12.1 grams protein/liter resin for MabSelect SuRe™ or 15.9 grams protein/liter resin for TOYOPEARL AF-rProtein A HC-650 shown in FIG. 6A. HMW=aggregates of MSD-21. HMW brackets indicate area where HMW elute from column.

FIG. 7 shows an SDS-PAGE analysis of apo Protein A ligands from their respective stock solutions run under non-reduced denatured conditions. The TOYOPEARL AF-rProtein A HC-650 (Tosoh) Protein A ligand's predominant molecular weight is approximately 38 kDa (Lanes 2 and 3). The MabSelect SuRe™ (Select; Lanes 4 and 5) maintain two significantly populated species at approximately 50 kDa and 28 kDa. Lane 1 contains the molecular weight marker.

FIG. 8A shows quantitation of residue specific effects from the NMR-based titrations using the anti-CTLA4 V_{HH}: MabSelect SuRe™ (Mab Select) Protein A ligand titration. The upper panel represents overall impact of ligand binding normalized over a titration point as the ratio of the peak amplitude for a given resonance (Iᵣₑₛ) and the maximum amplitude of all peaks within a 2D [¹H^{N}, ¹⁵N]-HSQC spectroscopy (Iₘₐₓ). The lower panel is another representation of the data which takes out the average effect of peak broadening due to large molecular weight complex formation. This analysis highlights regions that are more greatly impacted upon complex formation and that can contribute to the binding surface. This feature can be calculated as taking the ratio between Iᵣₑₛ and the average of all Iᵣₑₛ [symbol] I_{conc} [symbol] ). The dashed line is the cutoff for residues that are considered to be significantly impacted by binding and is calculated as [symbol] Iᵣₑₛ/ [symbol] I_{conc} [symbol] −σ_{I,conc}, σ_{I,conc} where is the standard deviation of all Iᵣₑₛ within a spectrum. Residues are significantly impacted wren their Iᵣₑₛ/ [symbol] I_{conc} [symbol] values are less than the dashed line. Black squares represent data points, across the primary sequence, for which data from the bound complex could not be ascertained or if they were severely overlapped during the titration.

FIG. 8B shows residues that are strongly impacted by binding are bolded across the printed primary sequences. The CDR regions are underlined. The numbers next to the primary sequences correspond to the sum of all residues that were bolded for a given titration. Boxes drawn in black guide the reader to the residues that form a common interaction site based on the count shown in FIG. 8C.

FIG. 8C shows the total count across the four titrations per residue is shown. Residues are counted to be part of the common interaction site if they were found to be significant to binding in three or more of the titrations.

FIG. 9A shows the structural imprint of binding surfaces from NMR-based titrations onto an NMR-based structural model of the anti-PD-1 V_{HH} domain that sense the interaction with the MabSelect SuRe™ (Mab Select) or TOYOPEARL AF-rProtein A HC-650 (Tosoh) Protein A ligand. Amino acid residues within the darkened area (indicated by circles) of the surface correspond to amino acid residues impacted by binding. A ribbon diagram of the V_{HH} is superimposed onto the surface representation.

FIG. 9B shows the structural imprint of binding surfaces from NMR-based titrations onto an NMR-based structural model of the anti-CTLA4 V_{HH} domain that sense the interaction with the Mab Select or Tosoh Protein A ligand. Amino acid residues within the darkened area (indicated by circles) of the surface correspond to amino acid residues impacted by binding. A ribbon diagram of the V_{HH} is superimposed onto the surface representation.

FIG. 9C shows the amino acid residues that sense the core residue interactions common to the Mab Select and Tosoh bindings. Amino acid residues within the darkened area (indicated by circle) of the surface of the anti-PD-1 V_{HH} structural model correspond to those amino acid residues that sense the core residue interactions. A ribbon diagram of the V_{HH} is superimposed onto the surface representation.

### DETAILED DESCRIPTION OF THE INVENTION

### Definitions

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

“Aggregate” as used herein refers to a high molecular weight species of the ABP monomer.

“Antibody” as used herein refers to a glycoprotein comprising either (a) at least two heavy chains (HCs) and two light chains (LCs) inter-connected by disulfide bonds, or (b) in the case of a species of camelid antibody, at least two heavy chains (HCs) inter-connected by disulfide bonds. Each HC is comprised of a heavy chain variable region or domain (V_{H}) and a heavy chain constant region or domain. In certain naturally occurring IgG, IgD, and IgA antibodies, the heavy chain constant region is comprised of three domains, C_{H}1, C_{H}2 and C_{H}3. In general, the basic antibody structural unit for antibodies is a tetramer comprising two HC/LC pairs, except for the species of camelid antibodies comprising only two HCs, in which case the structural unit is a homodimer. Each tetramer includes two identical pairs of polypeptide chains, each pair having one LC (about 25 kDa) and HC chain (about 50-70 kDa).

In certain naturally occurring antibodies, each light chain is comprised of an LC variable region or domain (V_{L}) and a LC constant domain. The LC constant domain is comprised of one domain, C_{L}. The human V_{H} includes six family members: V_{H}1, V_{H}2, V_{H}3, V_{H}4, V_{H}5, and V_{H}6; and the human V_{L} includes 16 family members: V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10. Each of these family members can be further divided into particular subtypes. The V_{H} and V_{L} domains can be further subdivided into regions of hypervariability, termed complementarity determining regions (CDRs), interspersed with regions that are more conserved, termed framework regions (FR). Each V_{H} and V_{L} is composed of three CDR regions and four FR regions, arranged from amino-terminus to carboxy-terminus in the following order: FR1, CDR1, FR2, CDR2, FR3, CDR3, FR4.

The variable regions of the heavy and light chains contain a binding domain comprising the CDRs that interacts with a target molecule or an antigen. The constant regions of the antibodies may mediate the binding of the immunoglobulin to host tissues or factors, including various cells of the immune system (e.g., effector cells) and the first component (C1q) of the classical complement system. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chethana, et al., (1989) Nature 342:878-883.

Typically, the numbering of the amino acids in the heavy chain constant domain begins with number 118, which is in accordance with the Eu numbering scheme. The Eu numbering scheme is based upon the amino acid sequence of human IgG₁ (Eu), which has a constant domain that begins at amino acid position 118 of the amino acid sequence of the IgG₁ described in Edelman et al., Proc. Natl. Acad. Sci. USA. 63: 78-85 (1969), and is shown for the IgG₁, IgG₂, IgG₃, and IgG₄ constant domains in Berwanger, et al., Ed. Ginetoux, Correspondence between the IMGT unique numbering for C-DOMAIN, the IMGT exon numbering, the Eu and Kabat numberings: Human IGHG, Created: 17 May 2001, Version: 8 Jun. 2016, which is accessible at www.imgt.org/IMGTScientificChart/Numbering/Hu_IGHGnber.html#r).

“Antibody fragment” or “Antigen binding fragment” as used herein refers to fragments of full-length antibodies, i.e. antibody fragments that retain the ability to bind specifically to the antigen bound by the full-length antibody but are less than full-length and which either lack an Fc domain in its entirety or lack those portions of the Fc domain that confer binding of the antibody to the FcγRs. Examples of antibody binding fragments include, but are not limited to, Fab, Fab′, F(ab′)₂, and Fv fragments; diabodies; scFv, V_{HH} fragments, and ISVDs.

“Antigen” refers to any substance (such as an immunogen or a hapten) foreign to a body that is capable of evoking an immune response in that body either alone or after forming a complex with a larger molecule (such as a protein) and that is capable of binding with a product (such as an antibody or T cell) of the immune response. The term antigen further includes any substance that can bind to an antigen-binding polypeptide regardless of whether the antigen-binding polypeptide was derived from the immunization of an animal with an antigen or from a library of antigen-binding polypeptides synthesized in silico.

“Antigen-binding polypeptide monomer” or “ABP monomer” as used herein, refers to an antigen-binding polypeptide comprising one or more ISVDs that bind an epitope of a target molecule other than an epitope on human serum albumin (HSA) or human transferrin. In particular embodiments, the ABP monomer includes a half-life extender, which may be an ISVD that binds an epitope on HSA. For an ABP monomer comprising two or more ISVDs, the ISVDs are provided as a fusion protein in which the ISVDs are covalently linked in tandem in a head-to-tail orientation in which the carboxy (C) terminus of one ISVD is directly linked to the amino (N) terminus of another ISVD (e.g., ISVD-ISVD-HSA binder) or indirectly linked to the amino (N) terminus of another ISVD by a polypeptide linker (e.g., ISVD-linker-ISVD-linker-ISVD). An ABP monomer targeting a single epitope of a target molecule may be monovalent (i.e., comprises a single ISVD targeting the epitope of a target molecule), or multivalent when comprising more than one ISVD and each ISVD targets the same epitope of the target polypeptide (e.g., a bivalent, trivalent, or tetravalent ABP monomer). A monovalent or multivalent ABP monomer may be referred to as a monospecific ABP monomer. In other embodiments, the ABP monomer is multispecific, which means it comprises at least two ISVDs, each ISVD binding an epitope on a different target molecule or different epitopes on the same target molecule. Depending on the number of different target molecules such ABP monomers may be bispecific, trispecific, or tetraspecific. When determining whether an ABP monomer is multivalent or multispecific, the presence or absence of an ISVD targeting an epitope on HSA or human transferrin is not considered.

“Breakthrough” as used herein refers to the volume at which a particular polypeptide that is applied to a chromatography column begins to elute off the column because the column has no more capacity to bind the polypeptide. The term “10% breakthrough” refers to the volume of a particular polypeptide that is the volume at which 10% of the particular polypeptide that is applied to a chromatography column begins to elute off the column.

“Chromatography” as used herein, refers to the separation of chemically different molecules in a mixture from one another by contacting the mixture with an adsorbent, wherein one class of molecules reversibly binds to or is adsorbed onto the adsorbent. Molecules that are least strongly adsorbed to or retained by the adsorbent are released from the adsorbent under conditions where those more strongly adsorbed or retained are not.

“Contaminant” refers to any foreign or objectionable molecule, particularly a biological macromolecule such as a DNA, an RNA, or a protein, other than the protein being purified that is present in a sample of a protein being purified. Contaminants include, for example, other host cell proteins from cells used to recombinantly express the protein being purified, proteins that are part of an absorbent used in an affinity chromatography step that may leach into a sample during prior affinity chromatography step, such as Protein A, and mis-folded variants of the target protein itself.

“Cytotoxic T lymphocyte-associated antigen-4,” “CTLA-4,” “CTLA4,” “CTLA-4 antigen” and “CD152” (see, e.g., Murata, Am. J. Pathol. 155:453-460 (1999)) are used interchangeably, and include variants, isoforms, species homologs of human CTLA-4, and analogs having at least one common epitope with CTLA-4 (see, e.g., Balzano, Int. J. Cancer Suppl. 7:28-32 (1992)). The complete CTLA-4 nucleic acid sequence can be found under GenBank Accession No. L15006.

“Fc domain”, or “Fc” as used herein is the crystallizable fragment domain or region obtained from an antibody that comprises the C_{H}2 and C_{H}3 domains of an antibody. In an antibody, the two Fc domains are held together by two or more disulfide bonds and by hydrophobic interactions of the C_{H}3 domains. The Fc domain may be obtained by digesting an antibody with the protease papain.

“Host cell proteins” or “HCP” include proteins encoded by the naturally-occurring genome of a host cell into which DNA encoding a protein that is to be purified is introduced. Host cell proteins may be contaminants of the protein to be purified, the levels of which may be reduced by purification. Host cell proteins can be assayed for by any appropriate method including gel electrophoresis and staining and/or ELISA assay, among others. Host cell proteins include, for example, Chinese Hamster Ovary (CHO) proteins (CHOP) produced as a product of expression of recombinant proteins.

“High molecular weight species” or “HMW species” as used herein refers to an association of at least two polypeptide monomers. The association may arise by any method including, but not limited to, covalent, non-covalent, disulfide, or nonreducible crosslinking.

“Humanization” (also called Reshaping or CDR-grafting) as used herein is a well-established technique for reducing the immunogenicity of monoclonal antibodies (mAbs) or ISVDs from xenogeneic sources and for improving the effector functions (ADCC, complement activation, C1q binding). The engineered mAb is engineered using the techniques of molecular biology, however simple CDR-grafting of the non-human complementarity-determining regions (CDRs) into human frameworks often results in loss of binding affinity and/or specificity of the original mAb or ISVD. In order to humanize an antibody or ISVD, the design for humanization includes variations such as conservative amino acid substitutions in residues of the CDRs, and back substitution of residues from the non-human mAb or ISVD into the human framework regions (back mutations). The positions can be discerned or identified by sequence comparison for structural analysis or by analysis of a homology model of the variable regions' three-dimensional structure. The process of affinity maturation has most recently used phage libraries to vary the amino acids at chosen positions. Similarly, many approaches have been used to choose the most appropriate human frameworks in which to graft the non-human CDRs. As the datasets of known parameters for antibody and ISVD structures increases, so does the sophistication and refinement of these techniques. Consensus or germline sequences from a single antibody of the framework sequences within each light or heavy chain variable region from several different human mAbs can be used. Another approach to humanization is to modify only surface residues of the non-human sequence with the most common residues found in human mAbs and has been termed “resurfacing” or “veneering.” Often, the human or humanized antibody or ISVD is substantially non-immunogenic in humans.

“Humanized ISVD” as used herein refers to forms of ISVDs that contain sequences from both human and non-human (e.g., Camelid) antibodies. In general, the humanized ISVD will comprise hypervariable loops that correspond to those of a non-human immunoglobulin such as a Camelid heavy chain antibody in which all or substantially all of the framework (FR) regions are those of a human immunoglobulin sequence.

“Immunoglobulin single variable domain” (also referred to as “ISV” or ISVD”), “single domain antibody fragment” (also referred to as “sdAb”), and “single domain antigen binding molecule” (also referred to as “sdAB”) are terms that may be used interchangeably to refer to an immunoglobulin variable domain (which may be heavy chain or light chain domain, including a V_{H}, V_{HH} or V_{L} domain) that can form a functional antigen binding site without interaction with another variable domain (for example, without a V_{H}/V_{L} interaction as is required between the V_{H} and V_{L} domains of conventional four-chain monoclonal antibody). As used herein, the ISVD lacks a constant domain or at least the Fc domain present in the constant domain. Examples of ISVDs include NANOBODIES™ (including a V_{HH}, a humanized V_{HH}, and/or a camelized V_{H} such as camelized human V_{H}'s), IgNAR domains, single domain antibodies such as dAb's™, which are V_{H} domains or derived from a V_{H} domain, or V_{L} domains or derived from a V_{L} domain. Unless explicitly mentioned otherwise herein, ISVDs that are based on and/or derived from heavy chain variable domains (such as V_{H} or V_{HH} domains) are generally preferred. Most preferably, unless explicitly indicated otherwise herein, an ISVD will be derived from a Camelid V_{HH}. An ISVD includes at least one or two CDRs, or more typically at least three CDRs.

“Lymphocyte-activation gene 3” or “LAG3” refers to a protein designated CD223 (cluster of differentiation 223), which in humans is encoded by the LAG3 gene. LAG3 is a cell surface molecule with diverse biologic effects on T cell function. It is an immune checkpoint receptor.

“NANOBODY” and “NANOBODIES” as used herein are registered trademarks of Ablynx N.V.

“PD-1” refers to the programmed Death 1 (PD-1) protein, an inhibitory member of the extended CD28/CTLA-4 family of T cell regulators (Okazaki et al., Curr. Opin. Immunol. 14: 391779-82 (2002); Bennett et al., J. Immunol. 170:711-8 (2003)). Other members of the CD28 family include CD28, CTLA-4, ICOS and BTLA. The PD-1 gene encodes a 55 kDa type I transmembrane protein (Agata et al., Intl. Immunol. 8:765-72 (1996)). Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43). PD-1 is known as an immunoinhibitory protein that negatively regulates TCR signals (Ishida, Y. et al., EMBO J. 11:3887-3895 (1992); Blank, C. et al., Immunol. Immunother. 56(5):739-745 (Epub 2006 Dec. 29)). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al., J. Mol. Med. 81:281-7 (2003); Blank et al., Cancer Immunol. Immunother. 54:307-314 (2005); Konishi et al., Clin. Cancer Res. 10:5094-100 (2004)). Immune suppression can be reversed by inhibiting the local interaction of PD-1 with PD-L1 or PD-L2; the effect is additive when the interaction of PD-1 with PD-L2 is blocked as well (Iwai et al., Proc. Nat'l. Acad. Sci. USA 99:12293-12297 (2002); Brown et al., J. Immunol. 170:1257-66 (2003)). “Programmed Death 1,” “Programmed Cell Death 1,” “Protein PD-1,” “PD-1” “PD1,” “PDCD1,” “hPD-1” and “hPD-1” are used interchangeably, and include variants, isoforms, species homologs of human PD-1, and analogs having at least one common epitope with PD-1. The complete PD-1 sequence can be found under GenBank Accession No. U64863.

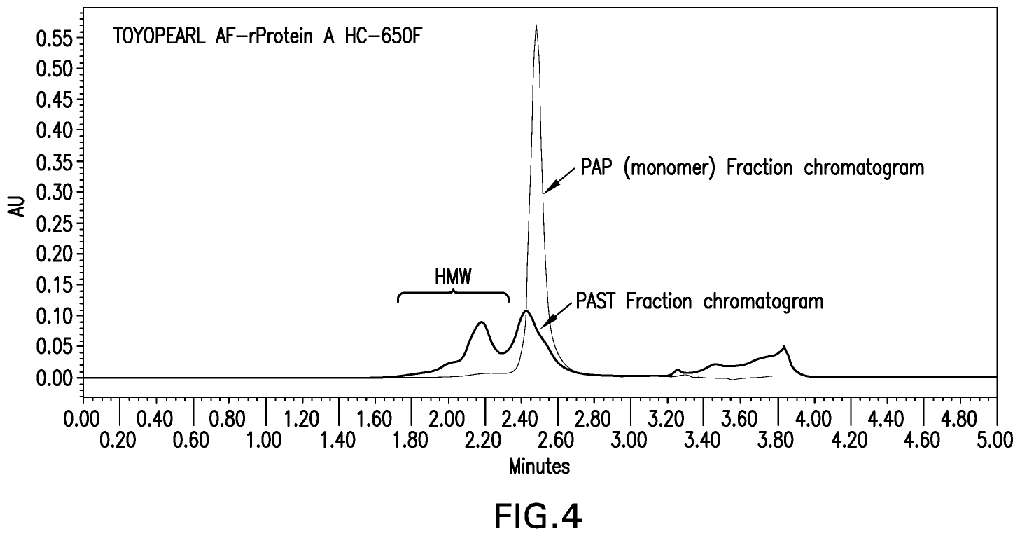

“Protein A” and associated phrases, such as “Protein A-based support” are intended to include Protein A (e.g., recombinant or isolated Protein A), or a functional variant thereof. In one embodiment, the Protein A is full length Staphylococcal Protein A (SpA) composed of five domains of about 50-60 amino acid residues known as E, D, A, B and C domains in order from the N-terminus. (Sjodhal Eur J Biochem 78: 471-490 (1977); Uhlen et al. J. Biol. Chem. 259: 1695-1702 (1984)). These domains contain approximately 58 residues, each sharing about 65%-90% amino acid sequence identity. Binding studies between Protein A and antibodies have shown that while all five domains of SpA (E, D, A, B and C) bind to an IgG via its Fc region, domains D and E exhibit significant Fab binding (Ljungberg et al. Mol. Immunol. 30(14):1279-1285 (1993); Roben et al. J. Immunol. 154:6437-6445 (1995); Starovasnik et al. Protein Sci 8:1423-1431 (1999). The Z-domain, a functional analog and energy-minimized version of the B domain (Nilsson et al. Protein Eng 1:107-113 (1987)) was shown to have negligible binding to the antibody variable domain region (Cedergren et al. Protein Eng. 6(4):441-448 (1993); Ljungberg et al. (1993) supra; Starovasnik et al. (1999) supra). Protein A can include the amino acid sequence of SpA (SEQ ID NO:11) shown in FIG. 4A, or an amino acid sequence substantially identical thereto. In other embodiments, the Protein A is a functional variant of SpA that includes at least one domain chosen from E, D, A, B and/or C, or a modified form thereof. For example, the functional variant of SpA can include at least domain B of SpA, or a variant of domain B, having one or more substituted asparagine residues, also referred to herein as domain Z. In one embodiment, the functional variant of SpA includes the amino acid sequence of SEQ ID NO:12) shown in FIG. 4B, or an amino acid sequence substantially identical thereto. Other permutations of functional variants of Protein A can be used comprising domain B, or a variant domain B, and one or more of: domains A and/or C; domains E, A and/or C; or domains E, D, A and/or C. Any combination of E, D, A, B and/or C, or a functional variant thereof, can be used as long as the combination is capable of binding to the ISVD.

“Purify” with respect to a polypeptide monomer means to reduce the amounts of foreign or objectionable elements, especially biological macromolecules such as proteins or DNA, that may be present in a sample of the protein. The presence of foreign proteins may be assayed by any appropriate method including gel electrophoresis and staining and/or ELISA assay. The presence of DNA may be assayed by any appropriate method including gel electrophoresis and staining and/or assays employing polymerase chain reaction. In embodiments, the polypeptide, e.g., the polypeptide monomer, is purified to at least 85%, 90%, 95%, 96%, 97%, 98%, 99% or higher purity.

“Substantially free of high molecular weight species” or “Substantially free of aggregates” is used herein to refer to compositions of ABP monomers in which less than 1% of the ABP monomers in the composition are in aggregates or high molecular weight species. The term “free of high molecular weight species” or “free of high molecular weight species” refers to compositions of ABP monomers in which less than about 5% of the ABP monomers in the composition are in aggregates or high molecular weight species.

“V_{HH}” as used herein indicates that the V_{H} domain is obtained from or originated or derived from a HC-only antibody. Heavy chain antibodies are functional antibodies that have two HCs and no LCs. Heavy chain antibodies exist in and are obtainable from Camelids, members of the biological family Camelidae.

### Protein A Chromatography

The present invention provides a process for isolating antigen-binding polypeptide (ABP) monomers comprising one or more immunoglobulin single variable domains (ISVDs) from aggregates (or high molecular weight species) of the ABP monomers that may be present in a harvested cell culture fluid (HCCF) obtained from cell cultures comprising cells expressing the ABP monomers. The method comprises applying the HCCF to a column comprising a Protein A resin selected from the group consisting of TOYOPEARL AF-rProtein A HC-650F resin or AMSPHERE A3 Protein A resin, each equilibrated in an equilibration solution at a neutral pH; washing the column with a wash solution at a neutral pH; and eluting the ABP monomers from the column with an elution solution at a pH of about 3.5 to obtain an eluent comprising the ABP monomers separated from aggregates of the ABP monomers.

Crystallographic data has shown that the primary binding site for protein A on an antibody is between the C_{H}2 and C_{H}3 domains within the antibody Fc domain. In addition, Sasso et al., J. Immunol. 147: 1877-1883 (1991) has shown that protein A can bind human IgG molecules containing IgG F(ab′)₂ fragments from the human V_{H}3 gene family, and Jansson et al. FEMS Immunol. Med. Microbiol. 20: 69-78 (1998) has shown that scFv and Fabs bind all domains of Protein A. Frenken et al., J. Biotechnol. 78:11-21 (2000) and Fridy et al., Nat. Meth. 11: 1253-1260 (2014) both show that Camelid heavy chain only antibodies can also be isolated using Protein A column chromatography. Frenken et al. op. cit. also showed that V_{HH} fragments can be isolated using the HITRAP Protein A Cartridge (Sigma-Aldrich, St. Louis, Mo.). U.S. Pat. No. 10,118,962 (prior U.S. Pat. Pub. 20100172894), WO2006122786 or WO2009068627 demonstrate use of MabSelect SuRe™ Protein A chromatography to isolate polypeptides comprising V_{HH} fragments under elution conditions comprising a glycine solution at pH 3.0 (U.S. Pat. No. 10,118,962) or 2.5, respectively. Fridy et al., Anal. Biochem. 477: 92-94 (2015) suggested that mutations of two residues in the V_{HH} fragment, Arg in position 21 (R21) and Asn in position 85 (N85), were required for Protein A binding. Positions R21 and N85 correspond to positions R19 and N82a according to Kabat numbering when the N-terminus begins with amino acid Asp (D) or Glu (E). More recently, Gerald et al., JSR Life Sciences (2017) provides a protocol for using AMSPHERE A3 Protein A chromatography to isolate antibody fragments and single domain antibodies (e.g., V_{HH} fragments and polypeptides comprising V_{HH} fragments) under elution conditions comprising a sodium acetate solution at pH 3.0. While the aforementioned do not disclose whether Protein A may be used to separate V_{HH} monomers from V_{HH} aggregates, U.S. Pat. No. 10,118,962 teaches using hydroxyapatite chromatography to remove aggregates and WO2009068627 teaches that V_{HH}s are highly soluble and do not have a tendency to aggregate.

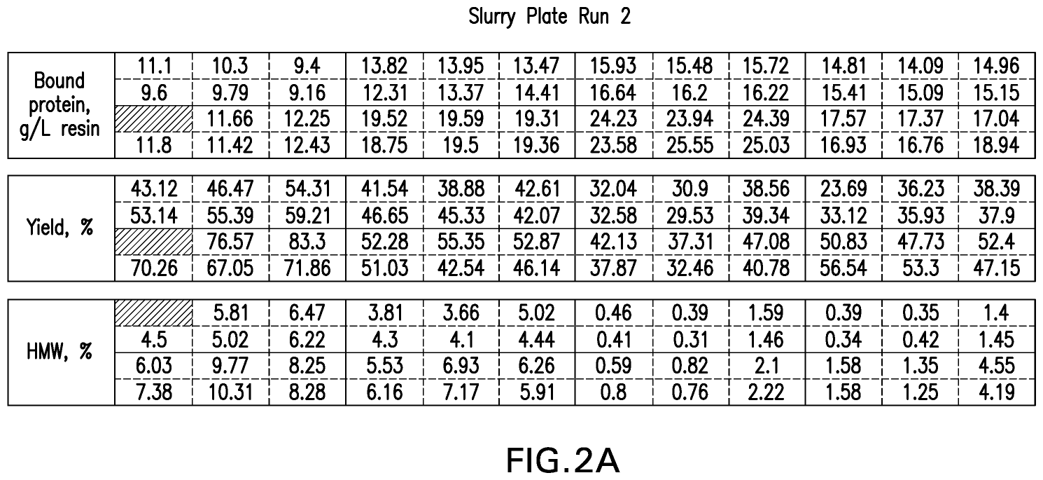

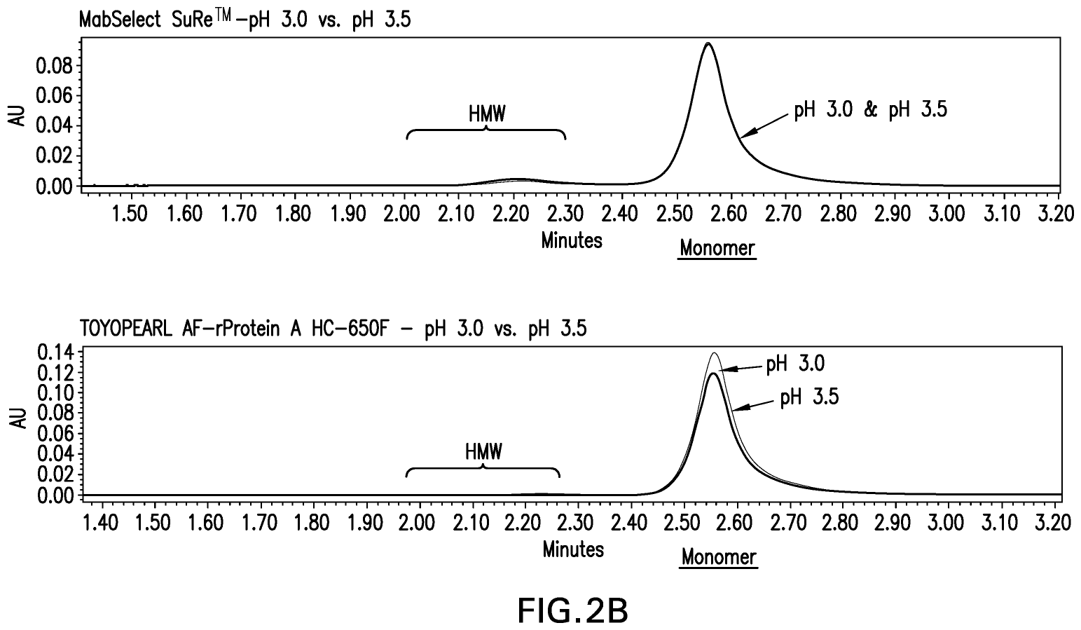

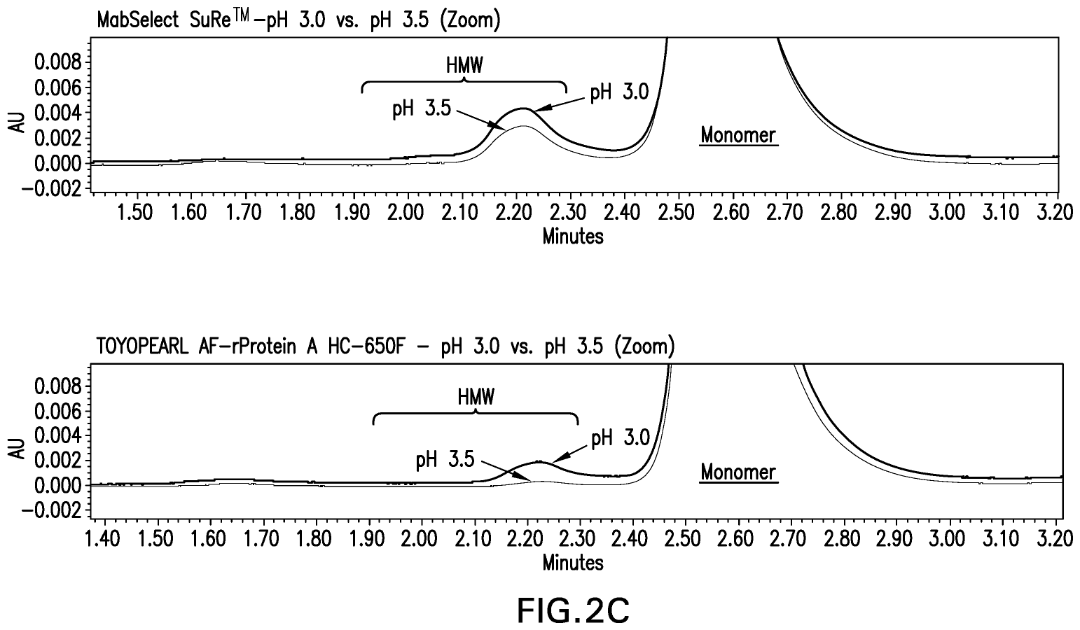

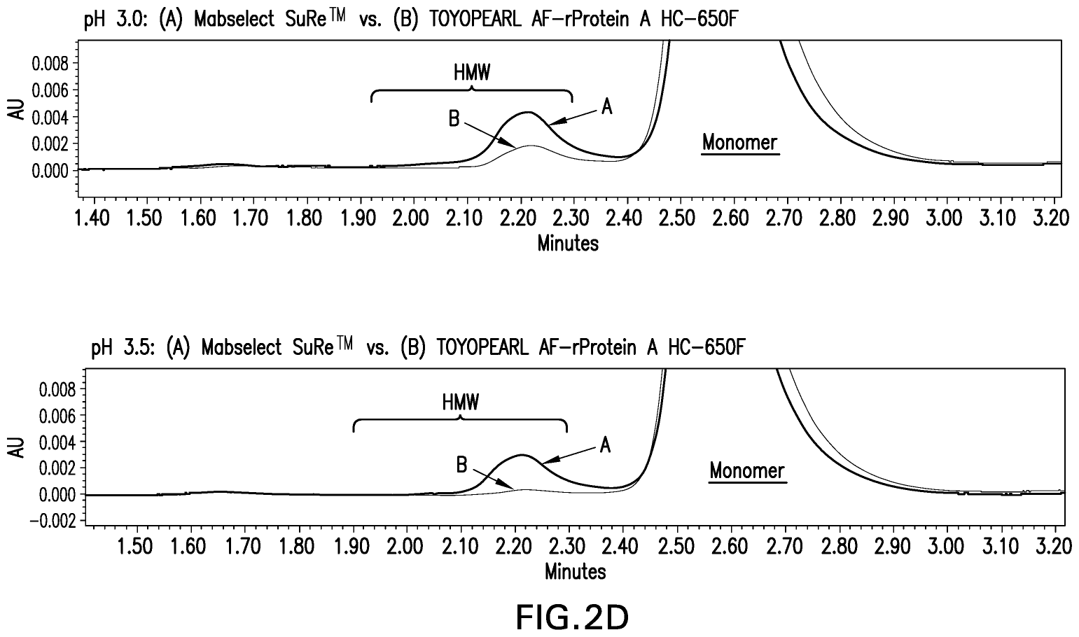

The inventors have discovered that Protein A chromatography may be used to separate ABP monomers, which comprise one or more ISVDs (e.g., VHH domains), from aggregates of the ABP monomers. The present invention is particularly useful for purifying ABP monomers that have a tendency to aggregate. The invention is based on the discovery that Protein A resins from different suppliers bind aggregates of ABP monomers to different extents or avidity, particularly during elution of the ABP monomers. The inventors found that compared to other commercially available Protein A resins, TOYOPEARL AF-rProtein A HC-650F or AMSPHERE A3 Protein A resins bind aggregates of the ABP monomers more strongly or with greater avidity than ABP monomers under the pH 3.5 elution conditions disclosed herein. Thus, during elution of the ABP monomers from the TOYOPEARL AF-rProtein A HC-650F or AMSPHERE A3 Protein A resins at pH 3.5, more of the aggregates of the ABP monomers will remain bound to either resin thus providing an eluant that comprises ABP monomers with less than 5% ABP monomer aggregates. As shown in FIG. 2A, elution of an exemplary ABP monomer from TOYOPEARL AF-rProtein A HC-650F or AMSPHERE A3 Protein A resins at pH 3.5 produced a composition of ABP monomers with less than about 2.0% aggregates compared to elution from two different MabSelect SuRe™ Protein A resins, which produced a composition of ABP monomers with about 3.6% to 5.8% aggregates. In particular embodiments as illustrated in the Examples, the process has enabled the production of compositions of ABP monomers with less than about 1.6% aggregates and on slurry plates, compositions of ABP monomers with less than about 0.5% aggregates have been obtained.

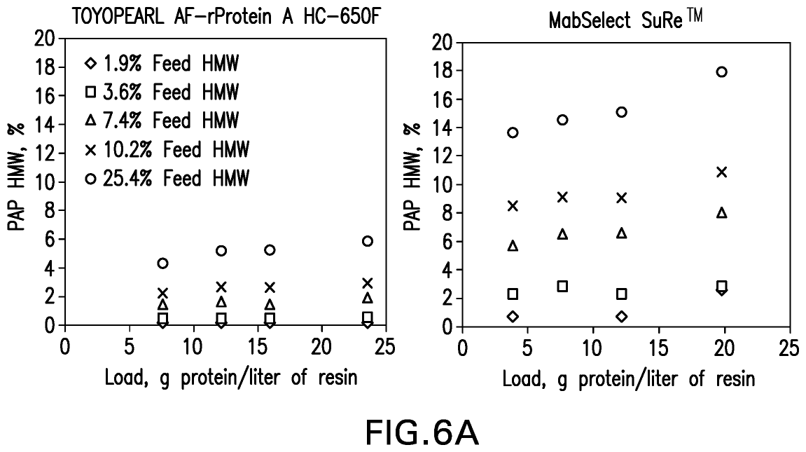

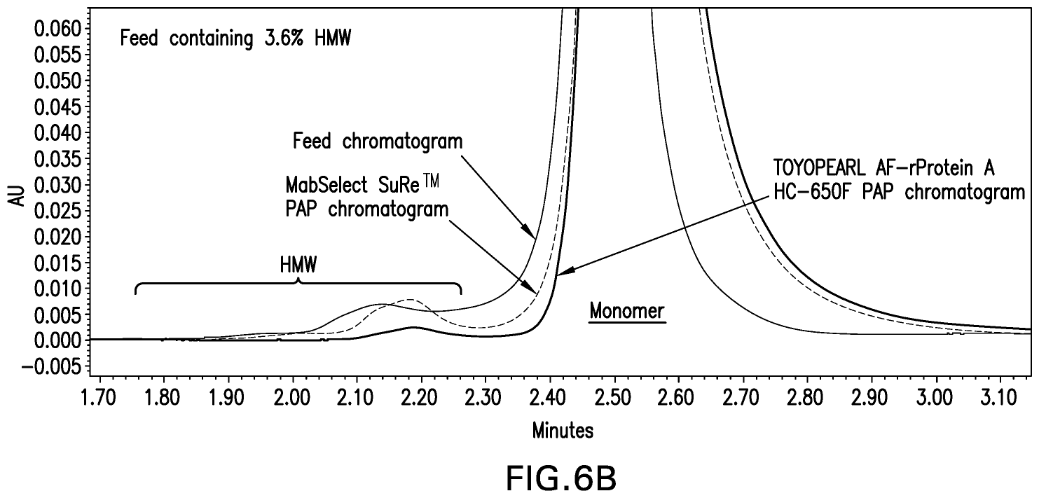

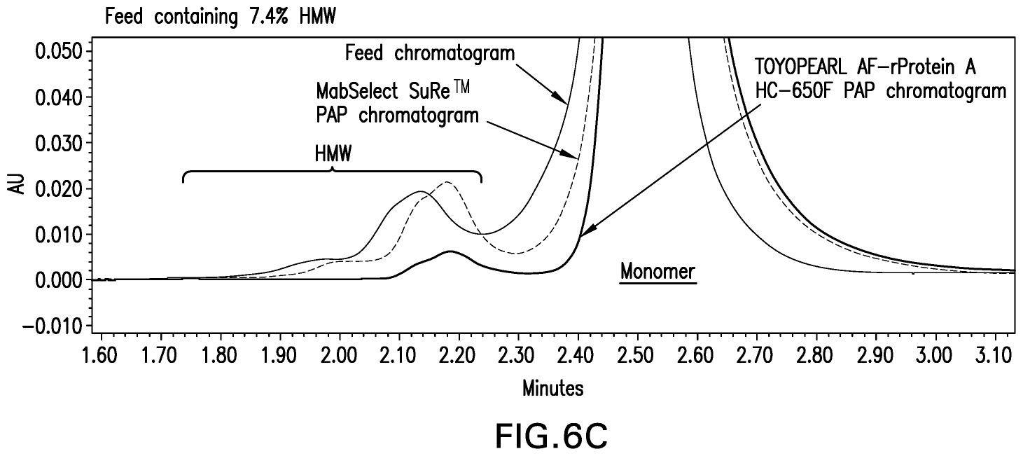

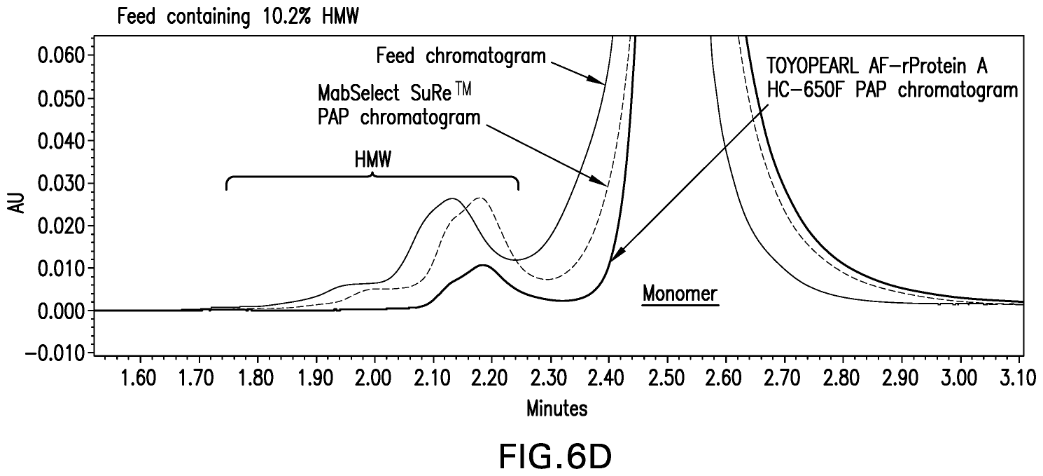

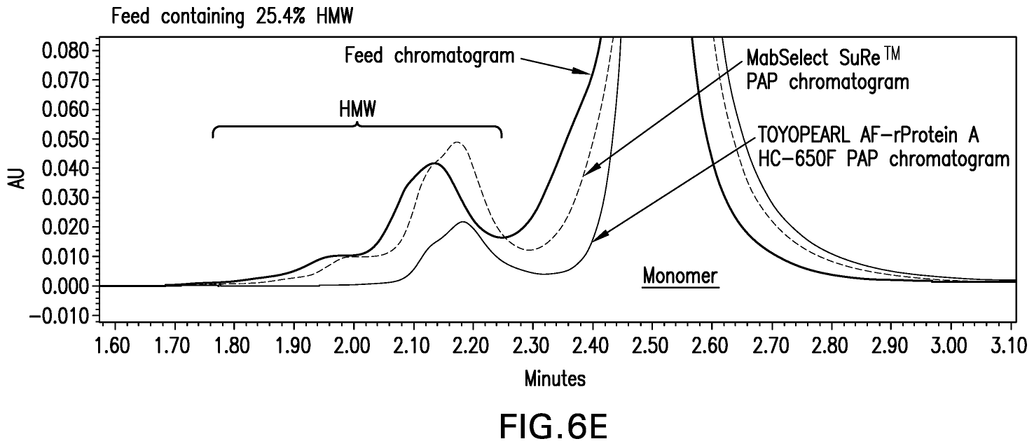

As illustrated in FIG. 6A, the process of the present invention provides that for a given range of feed HMW applied to the column, the aggregate amount in the eluant will be about three to five-fold lower than the amount of aggregate in eluant obtained using MabSelect SuRe™.

Therefore, while the HCCF comprises ABP monomers in a mixture with aggregates of the ABP monomers and other contaminants from the cell culture fermentation process, the Protein A chromatography method of the present invention enables the separation of the ABP monomers from aggregates of the ABP monomers to provide a composition that is substantially free of aggregates of the ABP monomers.

In particular embodiments, the composition comprises ABP monomers and less than about 2% aggregates of the ABP monomers as may be obtained when the protein load applied to the Protein A chromatography column is about 20 grams protein/liter resin or less.

In particular embodiments, the composition comprises ABP monomers and less than about 0.5% aggregates of the ABP monomers as may be obtained when the protein load applied to the Protein A chromatography column is about 20 grams protein/liter resin or less.

In particular embodiments, the composition comprises ABP monomers and less than about 1.6% aggregates of the ABP monomers as may be obtained when the protein load applied to the Protein A chromatography column is greater than about 20 grams protein/liter resin.

In particular embodiments, the composition comprises ABP monomers and less than 5% aggregates of the ABP monomers as may be obtained when the protein load applied to the Protein A chromatography column is about 40 grams protein/liter resin. The composition comprises ABP monomers and less than 2% aggregates of the ABP monomers.

The present invention provides a process for separating antigen-binding polypeptide (ABP) monomers from aggregates of the ABP monomers in a harvested cell culture fluid (HCCF), comprising (a) providing a HCCF from a culture of recombinant cells expressing ABP monomers, wherein the total protein in the HCCF comprises a mixture of the ABP monomers, aggregates of the ABP monomers, and other protein, and a chromatography column comprising TOYOPEARL AF-rProtein A HC-650F resin or AMSPHERE A3 resin, each equilibrated in an equilibration solution at a slightly acidic pH or neutral pH; (b) applying the HCCF to the chromatography column; (c) washing the chromatography column with at least one wash solution at a neutral pH or slightly acidic pH; and (d) eluting the polypeptide monomers from the chromatography column with an elution solution at about pH 3.5 to obtain an eluent comprising the ABP monomer, wherein the eluant comprises less than 5% (or in particular embodiments, less than 2%) of the aggregates of the ABP monomers as determined by ultra-performance size exclusion chromatography and wherein an ABP monomer comprises one or more immunoglobulin single variable domains (ISVDs).

As used in the present invention, a slightly acidic pH is a pH that is greater than about pH 6.0 and less than pH 7.0 and a neutral pH is a pH of 7.0 to about 7.5 pH units. In particular embodiments, slightly acidic pH is about pH 6.5. The inventors have found that the HCCF usually has a slightly acidic pH but will, if needed, adjust the pH to about 6.5 pH units.

In general, the HCCF is obtained from mammalian or yeast cell cultures comprising cells genetically modified to produce recombinant ABP monomers, which are secreted into the culture fluid. The HCCF obtained from the cell culture without further modification except adjustment as needed of the pH to a slightly acidic pH and any adjustment to the protein concentration to bring the total protein concentration to about 9 to 18 grams protein/liter resin is applied to the Protein A resin.

In a further embodiment, the HCCF is applied to the Protein A column at a continuous flow rate until the amount of total protein applied to the column reaches an amount that has been predetermined to be about 10% breakthrough or the amount of total protein applied to the column is about 9 to 18 grams protein/liter resin. In particular aspects, the total protein concentration of the HCCF comprises about 1.0 grams/liter to about 1.5 grams/liter of protein. In particular embodiments, the HCCF comprises about 9 grams protein/liter resin to about 18 grams protein/liter resin, which in further aspects may be applied to the chromatography column at a flow rate of up to about 300 cm/hour and/or up to at least 10% breakthrough.

In a further embodiment of the process, the equilibration solution and the at least one wash solution comprises Tris, glycine, or sodium phosphate and optionally a salt, e.g., sodium chloride. In particular embodiments, the equilibration solution and the at least one wash solution, each comprises about 10 to 20 mM sodium phosphate or 10 to 50 mM Tris. In particular embodiments, the equilibration solution and the at least one wash solution, each has a pH of about pH 6.5. In particular embodiments, the column is washed with two wash solutions: a first wash solution at about pH 6.5 comprising of sodium phosphate and a second wash solution at about pH 6.5 comprising of sodium phosphate and sodium chloride. In a further embodiment, the first and second wash solutions comprise about 10 to 20 mM sodium phosphate and the second wash solution further comprises about 500 mM sodium chloride. In a further still embodiment, the first and second wash solutions comprise about 10 mM sodium phosphate and the second wash solution further comprises about 500 mM sodium chloride. In a further embodiment, the column is washed with the first wash solution, then washed with the second wash solution, and finally washed with the first wash solution. In particular embodiments, the column is washed with about three column volumes (CVs) of the first wash solution, then washed with about five CVs of the second wash solution, and finally washed with about five CVs of the first wash solution.

In a further embodiment of the process, the ABP monomers are eluted from the column with an elution solution comprising sodium acetate and having a pH of about 3.5 pH units. In particular aspects, the elution solution comprises sodium acetate at a concentration of about 10 to about 100 mM. In further embodiments, the sodium acetate may be at a concentration of about 20 to about 100 mM, or in particular embodiments at a concentration of about 20 mM or about 100 mM. The elution may be performed by measuring the absorbance at 280 nm (A₂₈₀) of the eluent at the column outlet and capturing the volume of eluant that has an absorbance at A₂₈₀ of about 0.25 absorbance units (AU) per centimeter (cm) to provide a product pool comprising the ABP monomers. Typically, the capture begins when the eluant at the column outlet has an A₂₈₀ of about 0.25 AU/cm and proceeds for about three to five column volumes to provide the product pool.

An exemplary process for practicing the present invention is shown in Table 1A.

TABLE 1A

| | | Solution | | | |
|---|---|---|---|---|---|
| Step | Solution | pH | Column Volume (CV) | | |
| Equili- | 10 mM | 6.5 ± 0.2 | 5 or more | | |
| bration | Sodium | | | | |
| | Phosphate | | | | |
| Load | HCCF | N.A. | N.A. | | |
| | (9-18 g | | | | |
| | protein/L | | | | |
| | resin) | | | | |
| Wash 1 | 10 mM | 6.5 ± 0.2 | 3 | | |
| | Sodium | | | | |
| | Phosphate | | | | |
| Wash 2 | 10 mM | 6.5 ± 0.2 | 5 | | |
| | Sodium | | | | |
| | Phosphate/ | | | | |
| | 0.5M NaCl | | | | |
| Wash 3 | 10 mM | 6.5 ± 0.2 | 5 | | |
| | Sodium | | | | |
| | Phosphate | | | | |
| Elution | 20 mM | 3.5 ± 0.1 | Action | A₂₈₀ | Total |
| | Sodium | | (CV) | (AU/cm) | Approxi- |
| | Acetate | | | | mate CV |
| | | | Peak Start | 0.25 | ≤8 |
| | | | ~1 | | |
| | | | Pool Size | N.A. | |
| | | | 3-5 | | |
| | | | Post Pool | 0.25 | |
| | | | 1-2 | | |

N.A. = not applicable
AU = absorbance units

In a further aspect of the process, following the Protein A chromatography, the eluant comprising the ABP monomers is held at around room temperature at about pH 3.5 for about 60 to 90 minutes. Afterwards, the pH of the eluant is adjusted to about pH 4.2 and then filtered through a depth filter in line with a 0.22 um filter to provide a filtered neutralized viral inactivated product (FNVP) comprising ABP monomers wherein less than 1% of the ABP monomers are in aggregates. In a further embodiment, the FNVP comprises ABP monomers wherein less than 0.5% of the ABP monomers are in aggregates. In particular aspects, the FNVP is an aqueous composition comprising (a) ABP monomers and less than 1% aggregates of the ABP monomers or (b) ABP monomers and less than 0.5% aggregates of the ABP monomers.

The FNVP may be stored or further purified in subsequent downstream polishing steps to provide an aqueous composition comprising the ABP monomer and a pharmaceutically acceptable carrier wherein the composition comprises less than about 5% aggregates of the ABP monomer. In particular embodiments, the composition comprises less than about 2% aggregates of the ABP monomer.

### Exemplary ISVDs

The present invention is useful for isolating or purifying ABP monomers from aggregates of said monomers wherein one or more of the ISVDs comprising the ABP monomer comprises a humanized Camelid variable heavy domain (V_{HH}), which in further embodiments comprise an arginine residue at position 19 and an asparagine residue at position 82a wherein the position numbers are according to Kabat. While the present invention is useful for purifying or isolating any ABP monomer comprising an amino acid sequence therein that can bind to Protein A, the present invention is further useful for isolating or purifying ABP monomers that can bind Protein A and have a tendency to form aggregates.

In particular embodiments, the ABP monomer comprises at least one ISVD that comprises an amino acid sequence that binds programmed death receptor 1 (PD-1). In a further embodiment, the ABP monomer comprises at least two ISVDs wherein one ISVD has an amino acid sequence that binds programmed death receptor 1 (PD-1) and the other ISVD or ISVDs have amino acid sequences that bind another antigens.

In a further embodiment of the process, the ABP monomers comprise (i) at least one ISVD amino acid sequence that binds PD-1 and at least one ISVD amino acid sequence that binds human serum albumin (HSA); (ii) at least one ISVD amino acid sequence that binds PD-1 and at least one ISVD amino acid sequence that binds lymphocyte activation gene 3 (LAG3); (iii) at least one ISVD amino acid sequence that binds PD-1, at least one ISVD amino acid sequence that binds LAG3, and at least one ISVD amino acid sequence that binds HSA; (iv) at least one ISVD amino acid sequence that binds PD-1 and at least one ISVD amino acid sequence that binds cytotoxic T-lymphocyte-associated protein 4 (CTLA4); or (v) at least one ISVD amino acid sequence that binds PD-1, at least one ISVD amino acid sequence that binds cytotoxic CTLA4, and at least one ISVD amino acid sequence that binds HSA.

Exemplary ISVDs that may be incorporated into an ABP monomer include anti-PD-1 ISVDs, anti-LAG3 ISVDs, anti-CTLA4 ISVDs, and anti-HSA ISVDs as shown in Table 1B.

TABLE 1B
Exemplary ISVD Sequences

| | |
|---|---|
| Anti- | [symbol] VQLVESGGG [symbol] VQPGGSL [symbol] LSCAASGSIA [symbol] HAMG [symbol] FR |
| PD1 | QAPGKEREFV [symbol] VT [symbol] SGGITYYADSVKG [symbol] FTISRD [symbol] K |
| (SEQ ID | NTVYLQ [symbol] EDTA [symbol] YYCAGD [symbol] HQS [symbol] |
| NO: 16) | [symbol] [symbol] GQGTVLTVS [symbol] |
| LAG3 | [symbol] VQLVESGGG [symbol] VQPGGSL [symbol] LSCAASGRTF [symbol] DYVMG [symbol] FR |
| (SEQ ID | QAPGKEREFVA [symbol] AI [symbol] SGGRTHYAD [symbol] VKG [symbol] FTISRD [symbol] |
| NO: 18) | KNTLYLQ [symbol] PEDTA [symbol] YYCAT [symbol] LWW [symbol] SEYA |
| | PIKANDY [symbol] [symbol] GGQGTLVTVSS |
| CTLA4 | [symbol] VQLVESGGG [symbol] VQPGGSL [symbol] LSCAAS [symbol] FYGMG |
| 011F01 | [symbol] FRQAPGKEREFV [symbol] DIR₅₂T₅₂ₐSAGRTYYADSVKG [symbol] FTISRDY |
| (SEQ ID | [symbol] KNTVYLQ [symbol] PED [symbol] A [symbol] YYCA [symbol] E [symbol] SGI |
| NO: 22) | [symbol] GW [symbol] [symbol] GQGTLVTVS [symbol] |
| Anti- | [symbol] VQLVESGGG [symbol] VQPGNSL [symbol] LSCAASGFTF [symbol] SFGMS [symbol] VR |
| HSA | QAPGKGLEWV [symbol] SI [symbol] SGSDTLYADSVKG [symbol] RD |
| (SEQ ID | [symbol] KTTLYLQ [symbol] PEDTA [symbol] YYCTIG [symbol] SLS [symbol] |
| NO: 20) | [symbol] SQGTLVTVS [symbol] |

Number shown is according to Kabat numbering.

In further embodiments of the process, the ISVD amino acid sequence that binds PD-1 is a camelid variable domain (V_{HH}) amino acid sequence comprising a complementarity determining region (CDR) 1 having the amino acid sequence set forth in SEQ ID NO: 1; a CDR 2 having the amino acid sequence set forth in SEQ ID NO: 2 or 3; and a CDR 3 having the amino acid sequence set forth in SEQ ID NO: 4 or 5.

In further embodiments of the process, the ISVD amino acid sequence that binds LAG3 is a camelid variable domain (V_{HH}) amino acid sequence comprising a complementarity determining region (CDR) 1 having the amino acid sequence set forth in SEQ ID NO: 6; a CDR 2 having the amino acid sequence set forth in SEQ ID NO: 7; and a CDR 3 having the amino acid sequence set forth in SEQ ID NO: 8.

In further embodiments of the process, the ISVD amino acid sequence that binds HSA is a camelid variable domain (V_{HH}) amino acid sequence comprising a complementarity determining region (CDR) 1 having the amino acid sequence set forth in SEQ ID NO: 13; a CDR 2 having the amino acid sequence set forth in SEQ ID NO: 14; and a CDR 3 having the amino acid sequence set forth in SEQ ID NO: 15.

In further embodiments of the process, the ISVD amino acid sequence that binds CTLA4 is a camelid variable domain (V_{HH}) amino acid sequence comprising a complementarity determining region (CDR) 1 having the amino acid sequence set forth in SEQ ID NO: 9; a CDR 2 having the amino acid sequence set forth in SEQ ID NO: 10; and a CDR 3 having the amino acid sequence set forth in SEQ ID NO: 11 or 12.

In further embodiments of the process, the amino acid sequence that binds PD-1 comprises the amino acid sequence set forth in SEQ ID NO: 16 or 17; the amino acid sequence that binds LAG3 comprises the amino acid sequence set forth in SEQ ID NO: 18 or 19; the amino acid sequence that binds HSA comprises the amino acid sequence set forth in SEQ ID NO: 20 or 21; and the amino acid sequence that binds CTLA4 comprises the amino acid sequence set forth in SEQ ID NO: 22 or 23.

In further embodiments of the process, the ABP monomer is multispecific polypeptide that binds PD-1 and LAG3 and comprises the amino acid sequence set forth in SEQ ID NO:24.

The following examples are intended to promote a further understanding of the present invention.

### GENERAL METHODS

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

### General Methods

### 1.1 Material

### 1.1.1 Harvested Cell Culture Fluid (HCCF)

HCCF batches were used for the development studies herein. Several purified materials with known compositions of HMW impurities were utilized in one of studies designed to investigate the effect of loading on HMW clearance throughout the Protein A unit operation. For this purpose, HCCF material was purified thorough the Protein A step and the Protein A product (PAP) pools and Protein A strip (PAST) pools were neutralized to pH 4.2 right away using 1M Tris buffer, analyzed by a UPSEC (ultra-performance sized exclusion chromatography) assay (See section 1.2.7.3) to determine the monomer purity, and frozen for later use.

Several solutions composed of different amounts of HMW species were prepared by mixing the appropriate amounts of generated PAP and PAST pools together. Concentrations of the prepared solutions then were adjusted to 1.0±0.1 mg/mL using 20 mM Na Acetate, pH 4.2 buffer. The pH was adjusted to 6.5 and filtered through 0.22 μm syringe filter just before loading. Two aliquots were taken from each solution before the loading step; one aliquot was immediately frozen, and the other aliquot was kept at room temperature during the run. HMW levels of the two aliquots for all the solutions were analyzed by UPSEC assay. This analysis was to investigate if there were any changes in feed quality due to the instability of material at load condition during the run that might impact the study results. The UPSEC analysis of these samples showed that no changes in quality of any of the load solutions occurred during the run due to the instability of those solutions.

### 1.1.2 Capture Chromatography Resins and Columns

A set of 10 Protein A affinity resins, listed in Table 2, were screened for use in the capture chromatography step for purifying exemplary ABP MSD-21. Exemplary ABP “MSD-21”, which has the amino acid sequence shown in SEQ ID NO: 24, is a fusion protein comprising in tandem an anti-PD1 ISVD, an anti-LAG3 ISVD, and an anti-HSA ISVD, each ISVD linked to the preceding ISVD by a GS35 polypeptide linker.

TABLE 2
Affinity Capture Chromatography Resins

| Resin | Manufacturer | Resin | Manufacturer |
|---|---|---|---|
| Mab Select SuRe ™ | GE Healthcare | CaptureSelect ™ | Thermo Fisher |
| | | IgG-CH₁ | Scientific |
| MabSelect SuRe ™ pcc | GE Healthcare | CaptureSelect ™ | Thermo Fisher |
| | | HSA | Scientific |
| Capto L ™ | GE Healthcare | CaptureSelect ™ | Thermo Fisher |
| | | fcXL | Scientific |
| AMSPHERE A3 | JSR Lifescience | ProSep ™-vA | EMD Millipore |
| | | Ultra | |
| TOYOPEARL AF-rProtein A | Tosoh Bioscience | Eshmuno A | EMD Millipore |
| HC-650F | | | |

Table 3 lists a set of PhyTip™ affinity capture columns that were used for investigation of the loading impacts on aggregate removal during Protein A process.

TABLE 3
PhyTip ™ Columns with Affinity Capture Resins

| | | | Column |
|---|---|---|---|
| | Resin | Column | Volume, |
| Affinity Resin | Manufacturer | Vendor | mL |
| Mab Select SuRe ™ | GE Healthcare | PhyNexus | 0.16 |
| TOYOPEARL AF-r Protein A | Tosoh | PhyNexus | 0.16 |
| HC-650F | Bioscience | | |

A set of column chromatography runs were conducted to determine the range of Protein A process parameters and conduct the small-scale developments. Table 4 lists the size of columns and their associated development studies.

TABLE 4
Chromatography Column Used throughout Process Development

| Column ID | Column Volume | Use |
|---|---|---|
| Atoll | 0.6 mL | Process parameters range |
| | | finding |
| HR 0.5 X 20 cm | 4 mL | DBC |
| Omnifit ® 0.66 X 16 cm | 5.5 mL | D.O.E |

### 2.2 Methods

Slurry plate screening initially was applied for assessing a variety of capture resins and elution buffer conditions in terms of static binding capacity, yield and high molecular weight (HMW) aggregate level in the Protein A product. PhyTip™ affinity capture columns were utilized to study the effect of loading and resin type on HMW removal across the Protein A step. Further optimization of Protein A process parameters was performed using Atoll and lab-scale column chromatography.

### 2.2.1 Slurry Plate Screening

Initial screening of Protein A resin and elution buffer candidates were conducted using slurry plate. Slurry plate runs were conducted using TECAN Evoware 2 software. Resins were equilibrated with 200 μL of 10 mM NaPO₄, pH 6.5 buffer for a total of 20 minutes, followed by loading 200 μL of HCCF into the wells which, depending on the amount of resin in the well, was equivalent to 20 and 40 g ISVD/L resin for each condition. Resins were in contact with the loads for 60 minutes. Afterwards, resins were washed through three cycles using 200 μL of 10 mM NaPO₄, pH 6.5, 10 mM NaPO₄, 0.5 M NaCl pH 6.5 and 10 mM NaPO₄, pH 6.5 respectively with 10 minutes contact time for each cycle. The captured proteins then were eluted by 200 μL of elution buffers and 60 min contact time. Liquid/resin separation in this experiment was done using 0.22 μm filter plate and vacuum filtration and mixing was performed using a shaker.

### 2.2.2 Atoll Column Screening

After the initial slurry plate screening, a range of column loadings and elution buffer conditions with the desired Protein A resin pre-packed into Atoll columns were investigated. Table 5 shows the steps included in these runs. Buffers used in these steps are same as what is listed in Table 16, unless otherwise specified in the text.

TABLE 5
Atoll Column-Scale Protein A Process Parameters

| Step | | Duration | Step | | Duration |
|---|---|---|---|---|---|
| # | Step | (CV) | # | Step | (CV) |
| 1 | Equilibration | 12 | 6 | Elution | Varied |
| 2 | Load | Varied | 7 | Strip | 6 |
| 3 | Wash 1 | 4 | 8 | Sanitization | 4 |
| 4 | Wash 2 | 4 | 9 | Storage | 4 |
| 5 | Wash 3 | 4 | | | |

### 2.2.3 Loading Effect Studies Using PhyTips™ Affinity Capture Columns

The impacts of Protein A loading and Protein A resin type on selectivity of resins toward the monomer versus aggregated species were assessed in two high throughput experiments using TECAN Evoware 2 software. PhyTip™ columns packed with 160 μL of desired Protein A resins were utilized in these experiments and discarded after eluting the products, except in one of the experiments (Run 2 in Table 12) where more captured material was eluted from the column and pooled separately using 0.1M acetic acid buffer. Buffers used in these experiments are as listed in Table 15. Buffers and Protein A loads were transferred into the separate 96-well plates (600 μL of each solution per PhyTip™ column per plate). Resins were exposed to the buffers and Protein A load by aspiration and dispensation of solutions, present in the wells, through the tip. Each PhyTip™ column was initially equilibrated with 2.4 mL of equilibration buffer (i.e., 4 plates), followed by loading the targeted amount of Protein A load. Subsequently, it was washed with 600 μL of each of the wash 1, wash 2 and wash 1 solutions, respectively (i.e. 1 plate per wash step). The loaded materials then were eluted using 6 plates of elution buffer and pooled together. The pH of pooled solutions ultimately were neutralized and samples were taken for the analytical purposes

### 2.2.4 Small Scale Protein A Chromatography

Protein A process parameters in small scale development works are listed in Table 15. Due to the different structure of ISVDs such as MSD-21 compared to traditional monoclonal antibody molecules, there are variations in some of the parameters than those used for purifying monoclonal antibodies.

Columns were packed using 150 mM NaCl packing buffer at 600 cm/hour, two times of the process flow rate, followed by packing quality assessment through pulse test using high salt concentration and measuring the HETP and asymmetry of the eluted salt peak. Packed columns were stored at 2-8° C. between experiments.

Column chromatography runs were performed on AKTA AVANT system controlled by Unicorn 6 software (GE Healthcare). Column operation followed the running parameters described in Table 15 unless otherwise specified by an experimental parameter.

### 2.2.4.1 Dynamic Binding Capacity (DBC)

MSD-21 harvested clarified culture fluid (HCCF) was loaded up to 30 grams protein/liter resin onto 4 mL columns of the screened resins. The flow through stream was fractionated and evaluated by analytical Protein A-HPLC assay for product concentration for measuring any protein breakthrough. Protein A product pool in each run was collected and analyzed for product quality assessments.

### 2.2.4.2 Design of Experiments (D.O.E)

A response surface methodology with central composite design using quadratic polynomial fitting and two blocks (two separate sets of a Protein A column and an AKTA AVANT instrument) were applied to screen the impact of the elution pH and column load on product quality and process performance parameters. D.O.E runs were executed using 5.5 mL columns with 16±1 cm bed height. Protein A product pools were immediately viral inactivated at pH 3.5 for 1 hour, followed by neutralization at pH 4.2 and 0.22 μm filtration to produce a filtered neutralized viral inactivated pool (FNVIP). Depth filteration was not applied for post-NVIP (neutralized viral inactivated pool) filtration in these experiments due to material limitations.

### 2.2.5 Viral Inactivation

In viral inactivation (VI) protein purification step, the Protein A pool is adjusted to a target pH with 1 M Acetic Acid, and maintained at this pH for an hour at a temperature of 20±4° C., to inactivate viruses that might be present. The hold time starts when the product pool is measured to be within the inactivation pH range. At the different study time points, the viral inactivated pool (VIP) is adjusted to a target pH of 4.2 with 1M Tris solution to obtain neutralized viral inactivated pool (NVIP), where samples were filtered through a 0.22 um filter for quality evaluation by UP-SEC and HP-IEX.

An extra set of development studies were conducted for investigation of stability of viral inactivation product at different inactivation pH and hold time conditions to investigate the impact of these parameters on product quality. Viral inactivation runs were performed at pH 3.4, 3.5, and 3.6 over a three-hour hold period. The samples were neutralized to pH 4.2 at each time and filtered through a 0.22 μm syringe filter for quality evaluation by UP-SEC and HP-IEX assays.

### 2.2.6 Analytical Assays

### 2.2.6.1 Protein A High-Performance Liquid-Chromatography (Protein A-HPLC)

Protein titer in HCCF samples were measured using an analytical Protein A-HPLC method. Samples were loaded into a HPLC column, packed with protein A affinity resins, followed by a wash step using 50 mM NaPO₄, 150 mM NaCl, pH 7.1. Then, the bound proteins were eluted using 12 mM HCl, 150 mM NaCl, pH 1.9. The elution profile was recorded using UV A-280 with the area of the elution peak converted to the protein concentration using a linear equation that had been developed specifically for MSD-21.

### 2.2.6.2 UV A280

Concentration of the Protein A product, VIP, NVIP, and FNVIP samples were measured via UV absorbance at a wavelength of 280 nm. Extinction coefficient for the ISVD is 1.90 (mL/mg cm).

### 2.2.6.3 Ultra-Performance Size-Exclusion Chromatography (UP-SEC)

Monomer purity is considered a critical quality attribute to therapeutic proteins. The UP-SEC test indicates the purity of the monomer within a given sample and quantifies the aggregates as early eluting peaks (e.g. soluble aggregates of antibodies) as well as the low molecular weight species as late eluting peaks (e.g. product fragments). The UP-SEC method was comprised of a Waters BEH200 column 4.6×150 mm analytical column, set-up on a Waters Acquity H-class Bio System (Waters). Monomer, dimer, and higher order aggregate separation was obtained in 50 mM phosphate, 450 mM arginine-HCl, pH 7.0 mobile phase at a flow rate of 0.5 mL/min for 5 minutes and a column temperature of 30° C.

UV280 nm absorbance was recorded during each injection and peaks were integrated using Empower software (Waters). The monomer purity percentage was determined by the monomer peak area divided by the total peak area. The aggregate content percentage was determined by the sum of the peak area of each aggregate peak divided by the total peak area.

### Example 1

This example illustrates the use of Protein A affinity chromatography to capture an exemplary ABP MSD-21 from the harvest stream while reducing the level of impurities such as media components, host cell protein (HCP) and nucleic acids, e.g., deoxyribonucleic acid polymers (DNA). The Protein A product obtained from the chromatography may subsequently be viral inactivated at low pH followed by neutralization. Ultimately, the neutralized pool may be filtered through a filtration series comprising a depth filter and a 0.22 μm sterile filter.

MSD-21 lacks an Fc domain, which makes for a different binding to the Protein A ligand compared to that of an antibody, which has an Fc component that is known to bind Protein A. This difference in Protein A binding required extensive resin screening to identify an effective capture step for purifying MSD-21 from HCCF. The small-scale development work, reported in this example, consisted of evaluation of dynamic binding capacity (DBC) of multiple Protein A resins and the optimization of the process for obtaining purified MSD-21 product, including viral inactivation to neutralize any viral contamination of the product and depth filtration of the neutralized viral-inactivated product. A set of Design of Experiment (DOE) robustness studies for the Protein A purification step identified TOYOPEARL AF-rProtein A HC-650Fresin as enabling purification of the product with the least amount of aggregation.

### 3.1 Affinity Capture Resin and Elution Buffer Screening

### 3.1.1 Screening Using Slurry Plate

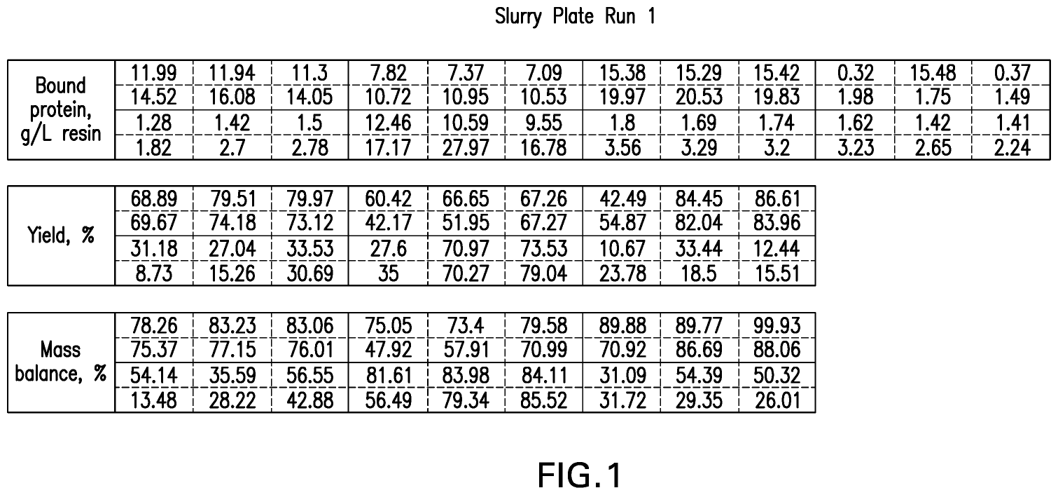

Slurry plate screening was conducted to assess the Protein A affinity resin and elution buffer conditions through analyzing loading capacity, yield and HMW aggregate level in the product. Initial screening of Protein A resin and elution buffer candidates were conducted using slurry plate. Table 6 shows the experimental design for Run 1 and the results are presented in FIG. 1 in the same format as the experimental design shown in Table 6.

TABLE 6
Design of Experiment - Slurry Plate Run 1

| | | | | | | | | | | | | |
|---|---|---|---|---|---|---|---|---|---|---|---|---|
| Load, | | | | | | | | | | | | |
| g/L | E1 | E2 | E3 | E1 | E2 | E3 | E1 | E2 | E3 | E1 | E2 | E3 |

| | | | | |
|---|---|---|---|---|
| 20 | Mab Select SuRe ™ | Eshmuno A | TOYOPEARL AF-r | CaptureSelect ™ IgG- |
| 40 | | | Protein A HC-650F | CH1 |
| 20 | Capto L ™ | ProSep ™-vA Ultra | CaptureSelect ™ | CaptureSelect ™ |
| 40 | | | fcXL | HSA |

E1 - elution solution comprising 5 mM Na Acetate, pH 3.5
E2 - elution solution comprising 5 mM Na Acetate, pH 3.0
E2 - elution solution comprising 5 mM Glycine, pH 3.2

FIG. 1 shows the capacity (grams bound protein/L resin) of each of the Protein A resins evaluated. Binding capacities of ProSep™ vA Ultra, MabSelect SuRe™ and TOYOPEARL AF-rProtein A HC-650F resins at different conditions were higher compared to the other evaluated resins with TOYOPEARL AF-rProtein A HC-650F resin having the highest binding capacities at both load conditions.

Yield and mass balance data of the evaluated resins, except for CaptureSelect HSA and CaptureSelect™ IgG-C_{H}1 resins, are shown in FIG. 1. MabSelect SuRe™ and TOYOPEARL AF-rProtein A HC-650F respectively resulted in higher process yields compared to ProSep™ vA Ultra resin at both load conditions and pH 3.5 elution buffer. Reducing the acetate buffer pH from 3.5 to 3.0 at low salt concentration (5 mM Na Acetate) caused an increase in product yield for all the resins. This increase was much more pronounced for TOYOPEARL AF-rProtein A HC-650F compared to MabSelect SuRe™ at both load conditions of 20 grams and 40 grams/liter resin. In addition, using glycine buffer at a low-pH did not make additional improvement in product recovery for both resins compared to the Na Acetate elution buffer.

Overall, TOYOPEARL AF-rProtein A HC-650F and MabSelect SuRe™, showed the highest yield and binding capacities among the other resins. Therefore, these two resins were chosen to be further investigated in another round of slurry plate screening.

The second round of slurry plate screening (Run 2) was performed using the experimental design shown in Table 7. The results are presented in FIG. 2 in the same format as the experimental design shown in Table 7.

TABLE 7
Design of Experiment - Slurry Plate Run 2

| | | | | | | | | | | | | |
|---|---|---|---|---|---|---|---|---|---|---|---|---|
| Load, | | | | | | | | | | | | |
| g/L | E4 | E5 | E6 | E4 | E5 | E6 | E4 | E5 | E6 | E4 | E5 | E6 |

| | | | | |
|---|---|---|---|---|
| 20 | Mab Select SuRe ™ | Mab Select SuRe ™ | AMSPHERE A3 | TOYOPEARL AF-r |
| 20 | | pcc | | Protein A HC-650F |
| 40 | Mab Select SuRe ™ | Mab Select SuRe ™ | AMSPHERE A3 | TOYOPEARL AF-r |
| 40 | | pcc | | Protein A HC-650F |

E4 - elution solution comprising 20 mM Na Acetate, pH3.5
E5 - elution solution comprising 100 mM Na Acetate, pH3.5
E6 - elution solution comprising 20 mM Na Acetate, pH3.0

As it is seen in FIG. 2A, overall, MabSelect SuRe™ gave the lowest binding capacity but the highest product recovery among the other resins. MabSelect SuRe™ pcc also showed 20 to 30% higher binding capacity than MabSelect SuRe™ resin. In addition, TOYOPEARL AF-rProtein A HC-650F and AMSPHERE A3 led to the highest binding capacity but the lowest process yields. FIG. 2B shows UP-SEC scans of the elution results shown in FIG. 2A comparing MabSelect SuRe™ eluants to TOYOPEARL AF-rProtein A HC-650F eluants. FIG. 2C is a zoom view of the scans in FIG. 2B and show that for TOYOPEARL AF-rProtein A HC-650F eluants, the amount of HBMW aggregates is significantly reduced under elution conditions performed at pH 3.5 compared to pH 3.0. FIG. 2D presents the zoom views of the MabSelect SuRe™ vs. TOYOPEARL AF-rProtein A HC-650F comparisons in overlays, which show that the present HMW aggregates are significantly reduced in the TOYOPEARL AF-rProtein A HC-650F eluants compared to MabSelect SuRe™ eluants. As shown, the TOYOPEARL AF-rProtein A HC-650F and AMSPHERE A3 eluants had significantly reduced HMW aggregation compared to the MabSelect SuRe™ eluants (e.g. 0.4% vs about 4% at 20 g/L load and 20 mM Na Acetate, pH 3.5 as the elution buffer). Increasing the elution buffer salt concentration to 100 mM had negligible impact on process yield and product aggregation for all the resins.

As shown in FIGS. 2A-2D, using a lower elution buffer pH for TOYOPEARL AF-rProtein A HC-650F and AMSPHERE A3 resins increased the aggregated product percentage. The lower elution pH had a negligible impact on the percentage of aggregated product obtained using MabSelect SuRe™-family resins. In addition, this reduction in pH didn't lead to a significant increase in process yield for all the resins.

In summary, the TOYOPEARL AF-rProtein A HC-650F and AMSPHERE A3 resins resulted in an MSD-21 product with less HMW aggregation than that obtainable using the MabSelect SuRe™ family of resins and the TOYOPEARL AF-rProtein A HC-650F and AMSPHERE A3 resins resulted in an MSD-21 product with significantly less HMW aggregation when the elution pH was 3.5 as opposed to elution at pH 3.0 as used in the art for eluting ISVD from Protein A.

### 3.1.2 Screening of Dynamic Binding Capacity (DBC)

The dynamic binding capacity of four Protein A affinity resins, as listed in Table 8, were evaluated up to a loading of 30 g of MSD-21/L resin.

TABLE 8
DBC-Screened Protein A Affinity Resins

| Resin | Manufacturer |
|---|---|
| Mab Select SuRe ™ | GE Healthcare ™ |
| Mab Select SuRe ™ pcc | GE Healthcare ™ |
| TOYOPEARL AF-rProtein A HC-650F | Tosoh Bioscience |
| AMSPHERE A3 | JSR Lifesciences |

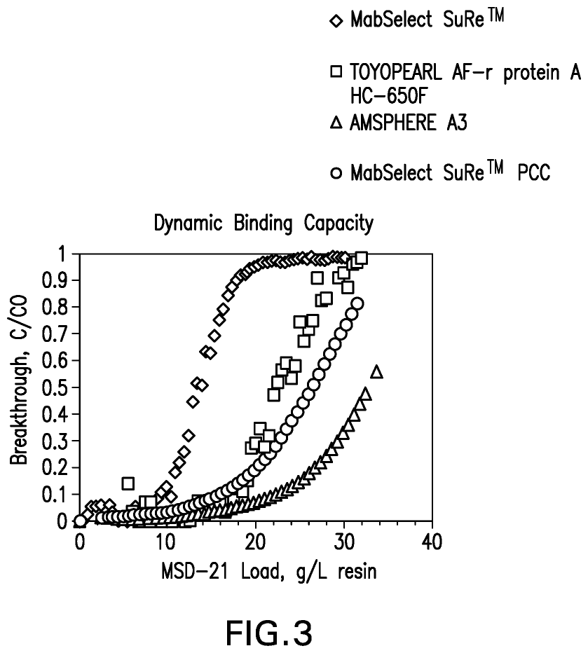

Fractions (1.6 mL, equivalent to 0.4 CV) were collected from the flow through during the run and analyzed later by Protein A-HPLC for any breakthrough trend. Protein A pool (PAP) collected in each run was analyzed as well for product quality. FIG. 3 shows the DBC profiles. The small bump initially seen in breakthrough is an artifact due to the lower accuracy of Protein A-HPLC assay at the lower end of concentrations. AMSPHERE A3 gave the highest protein binding capacity at 10% breakthrough (about 22 g protein/L resin). MabSelect Sure™ pcc and TOYOPEARL AF-rProtein A HC-650F had similar range of binding capacity at 10% breakthrough (i.e. 16-18 grams protein/liter resin), however TOYOPEARL AF-rProtein A HC-650F resulted into higher level of HMW clearance in Protein A pool compared to MabSelect SuRe™ pcc which was aligned with what was seen in FIG. 2. In addition, because it was recommended by GE Healthcarem that MabSelect SuRe™ pcc to pack in shorter bed height columns to prevent high pressure artifacts due to the smaller bead size of this resin compared to that of the MabSelect SuRe™, the amount of protein that could be loaded per cycle for both of the MabSelect™-family resins would be expected to be similar.

### 3.1.3 Screening Using Atoll Columns

After the initial slurry plate screening, a range of column loadings and elution buffer conditions with the desired Protein A resin pre-packed into Atoll columns were investigated. Table 9 shows the steps included in these runs. Buffers used in these steps are same as is listed in Table 16, unless otherwise specified in the text.

TABLE 9
Atoll Column-Scale Protein A Process Parameters

| | | Duration, | | | Duration, |
|---|---|---|---|---|---|
| Step # | Step | CV | Step # | Step | CV |
| 1 | Equilibration | 12 | 6 | Elution | Varied |
| 2 | Load | Varied | 7 | Strip | 6 |
| 3 | Wash 1 | 4 | 8 | Sanitization | 4 |
| 4 | Wash 2 | 4 | 9 | Storage | 4 |
| 5 | Wash 3 | 4 | | | |

In the first Atoll column screening run, a set of elution buffers was investigated using 0.6 mL Atoll column of MabSelect SuRe™ resin with 10 g MSD-2L resin load for all the conditions. Elution for all the runs was 10 column volumes (CV).

Table 10 summarizes the tested conditions and results. Although all the acetate buffers led to the same range of process yields (70-75%), the 20 mM acetate pH 3.5 buffer resulted in the lowest aggregate level in the Protein A product (PAP) pool. The addition of arginine increased the product recovery; however, it also resulted in significantly higher aggregated product compared to the 20 mM acetate pH 3.5 buffer.

TABLE 10
Protein A Elution Buffer Screening using MabSelect SuRe ™ Atoll Column

| | PAP Pool | PAP Pool | Step | | | |
|---|---|---|---|---|---|---|
| | Concentration | Volume | Yield | Aggregate, | Monomer | LMW |
| Elution buffer | (g/L) | (mL) | (%) | (%) | (%) | (%) |
| 20 mM Na Acetate, | 2.65 | 1.6 | 70.6 | 4.32 | 95.68 | 0 |
| pH 3.5 | | | | | | |
| 5 mM Na Acetate, | 1.72 | 2.6 | 74.5 | 9.42 | 90.58 | 0 |
| pH 3.5 | | | | | | |
| 5 mM Na Acetate, | 2.82 | 1.6 | 75.3 | 6.27 | 93.73 | 0 |
| pH 3.1 | | | | | | |
| 0.1M Arginine, | 5.16 | 1.4 | 120.4 | 10.01 | 89.99 | 0 |
| ~0.5M Acetate, | | | | | | |
| pH 3.5 | | | | | | |

The impact of column loading and type of Protein A resin on product quality and process performance was further investigated through another set of Atoll column chromatography runs with four replicates for each condition. Eight column volumes of 20 mM sodium acetate, pH 3.5, was used as the elution buffer in these runs.

Table 11 shows the averaged values of data for these experiments. Increasing the load had a significant impact on HMW aggregate level in the PAP pool when MabSelect SuRe™ was used; however, this impact was much less using TOYOPEARL AF-rProtein A HC-650F. Yield values were slightly lower for the TOYOPEARL AF-rProtein A HC-650F independent of loading.

TABLE 11
Protein A Column Load and Resin Screening by Atoll Column

| | | | PAP Pool | | |
|---|---|---|---|---|---|
| | Load, | PAP Pool | Concentra- | Yield, | PAP pool |
| Resin | g/L | Volume, mL | tion, g/L | % | HMW, % |
| MabSelect | 9.3 | 1.65 | 3.13 | 90 | 2.71 |
| SuRe ™ | 13.8 | 1.70 | 4.43 | 90 | 4.50 |
| TOYOPEARL | 6.9 | 2.75 | 1.14 | 75 | 0.75 |
| AF-r Protein A | 11.1 | 2.85 | 1.78 | 76 | 0.98 |
| HC-650F | | | | | |

These values for MabSelect SuRe™ were unexpectedly high. Furthermore, using TOYOPEARL AF-rProtein A HC-650F led to about 75% higher elution pool volume than using MabSelect SuRe™. The lower process yield by the TOYOPEARL AF-rProtein A HC-650F resin can be to some extent attributed to its better removal of HMW matter than MabSelect Sure. The significant variations observed in process performance parameters (yield and pool volume) and MSD-21 aggregation level obtained using TOYOPEARL AF-rProtein A HC-650F and MabSelect SuRe™ are related to the difference in structural specifications of the resins. To further evaluate these observations, a set of high throughput PhyTip™ column experiments was conducted to gain a better understating of the roles the loading conditions and Protein A resin type on the product aggregation level and process yield. (See section 2.1.4).

### 3.1.4 Investigation of Loading and Load Aggregate Level Effects Using PhyTips Columns

The impacts of Protein A loading and Protein A resin type on selectivity of resins toward the MSD-21 monomer versus aggregated MSD-21 were assessed in two experiments. PhyTip™ columns packed with 160 μL of desired Protein A resins were utilized in these experiments and discarded after eluting the products, except in one of the experiments (Run 2 in Table 12) where more captured material was eluted from the column and pooled separately using 0.1M acetic acid buffer. Buffers used in these experiments are as listed in Table 16.

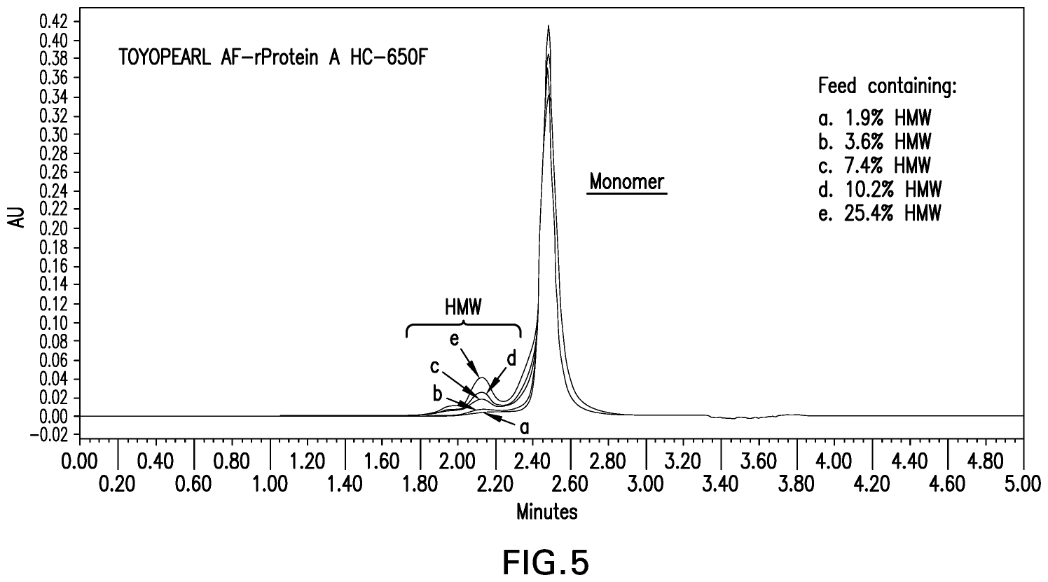

The impacts of loading and load aggregate level on product quality and yield obtained from columns using TOYOPEARL AF-rProtein A HC-650F resin were assessed. Each condition was duplicated in these experiments. Different levels of the feed aggregation were obtained by mixing the proper amounts of the generated PAP (Protein A POOL) and PAST (Protein A strip) fractions that had contained 2.44% and 44.96% HMW, respectively. FIG. 4 represents the overlay of UP-SEC chromatograms of PAP and PAST and FIG. 5 presents the overlay of UP-SEC chromatograms of prepared solutions spiked with different amounts of MSD-21 aggregate (HMW). The % HMW in the feed was 1.9%. 3.6%, 7.4%, 10.2%, and 25.4%. For more details on feed preparation (see section 1.1.1). FIG. 5 shows that the amount of HMW material in the PAP increased as a function of the amount of HMW in the feed but as shown below and in FIGS. 6A-6E, the increase is substantially less using the TOYOPEARL AF-rProtein A HC-650F resin than the increase observed using the MabSelect SuRe™ resin. A summary of conditions is shown in Table 12. The purity of the MSD-21 product pools was assessed using UP-SEC method.

TABLE 12
Protein A Loading Effect Study

| | | | Loading Range, |
|---|---|---|---|
| | | | grams protein/ |
| Run | Resin | Loading Material | liter resin |
| 1 | MabSelect SuRe ™ | HCCF | 5.3-21.2 |
| 2 | MabSelect SuRe ™ | Mix of PAP and | 3.8-19.7 |
| | TOYOPEARL AF-rProtein | PAST | 7.6-23.5 |
| | A HC-650F | | |

In order to have a better understanding of the impact of loading on yield and purity, Protein A resin type and feed aggregation level on the yield and capability of MSD-21 aggregate (HMW) clearance during Protein A step, another PhyTip™ column experiment was conducted (Run 2 in Table 12). In this run, PhyTip™ columns of both MabSelect SuRe™ and TOYOPEARL AF-rProtein A HC-650F and five solutions of purified materials composing of a range of known aggregation levels were used (See 1.1.1). Four different loadings were applied for each resin with each condition duplicated. FIG. 6A shows the percent MSD-21 aggregates (HMW) in the Protein A product pools (PAPs). As observed, and previously shown in Table 11, TOYOPEARL AF-rProtein A HC-650F provides greater HMW clearance compared to MabSelect SuRe™ at all the ranges of feed HMW amounts and loading values that were tested. For example, applying load material (feed) adjusted to contain 25.4% HMW onto TOYOPEARL AF-rProtein A HC-650F resin at 15.9 grams protein/liter resin, which is close to the dynamic binding capacity (DBC) of the resin, resulted in a 20.1% reduction of HMW in the PAP. However, applying a feed with the same % HMW onto MabSelect SuRe™ resin at 12 grams protein/liter resin, which is close to the DBC of the resin, resulted in a 10.3% reduction of HMW in the PAP. In addition, the data also indicates that for a given range of feed HMW amount (FIG. 6A), the % HMW in the PAP obtained using MabSelect SuRe™ is about three- to five-fold higher than the % HMW in the PAP obtained using TOYOPEARL AF-rProtein A HC-650F. FIGS. 6B-6E show the chromatograms for feeds containing 3.6%, 7.4%, 10.2%, and 25.4% HMW and PAP from columns in which the feed was loaded onto the column at a gram protein/liter resin concentration at about the DBC for the resin. The results are also tabulated in Table 13 and show that the % HMW is significantly less in PAP from the TOYOPEARL AF-rProtein A HC-650F compared to PAP from the MabSelect SuRe™, regardless of how much HMW is in the feed.

TABLE 13

| | | % HMW | % HMW | % HMW | % HMW in | % HMW in |
|---|---|---|---|---|---|---|
| | | in PAP | in PAP | in PAP | PAP from | PAP from |
| | Feed | from Feed | from Feed | from Feed | Feed with | Feed with |
| | (g protein/ | with 1.9% | with 3.6% | with 7.4% | 10.2% | 25.4% |
| | L resin) | HMW | HMW | HMW | HMW | HMW |
| Mabselect | 12.1 | 0.65 | 2.31 | 6.6 | 9.08 | 15.11 |
| SuRe ™ | | | | | | |
| TOYOPEARL | 15.9 | 0.21 | 0.46 | 1.5 | 2.62 | 5.31 |
| AF-rProtein A | | | | | | |
| HC-650F | | | | | | |

To elute the captured material (e.g., MSD-21 aggregates) following elution of the MSD-21 product, a strip step was conducted using 0.1M acetic acid. More material (about 10-25%) could be recovered from the TOYOPEARL AF-rProtein A HC-650F and about less than 1% for MabSelect SuRe™. Since the load materials used in this experiment were purified materials that had been recovered from TOYOPEARL AF-rProtein A HC-650F using the similar elution and strip buffers, a full recovery of these materials was expected after elution and strip steps in this experiment as well. However, about 25-40% of the loaded materials remained unrecoverable after elution and strip steps for both resins. This loss of material is probably due to a specific type of interaction between the MSD-21 and resin (combination of Protein A ligands and resins backbone) that the current buffer conditions are unable to modulate.

Overall, the data shows that across the evaluated ranges of loadings and load material aggregate levels, TOYOPEARL AF-rProtein A HC-650F provided better aggregate removal but lower product recovery yield than MabSelect SuRe™. Furthermore, the performance of the TOYOPEARL AF-rProtein A HC-650F was more robust and less impacted by the amount and quality of load material than the MabSelect SuRe™ resin. In conclusion, Protein A chromatography purification of MSD-21 using TOYOPEARL AF-rProtein A HC-650F and elution with 20 mM sodium Acetate, pH 3.5, provided a product of high purity.

### 3.1.5 Modification of Wash and Elution Steps Duration

The duration of the wash 2 and wash 3 steps were optimized through a set of experiments to ensure further removal of impurities in the wash 2 step and reduction of UV signal to the baseline in the wash 3 step before starting eluting the product from column. Ultimately, it was chosen to apply five column-volumes (CV) of each of wash 2 and wash 3 steps followed by auto-zeroing the UV signal at the beginning of elution step.

In addition, it was observed that the protein A product pool volume using the TOYOPEARL AF-rProtein A HC-650F was in the range of three to five column volumes using elution buffer (20 mM Na acetate, pH 3.5), which was larger than the PAP column volume in a typical monoclonal antibody purification process (two to three CV). Therefore, the elution step was extended to eight CV to ensure completed elution of MSD-21 product before stripping the column with low pH solution (See Table 16).

### 3.1.6 Range Finding Studies Via D.O.E

The Protein A chromatography D.O.E experiments for MSD-21 were performed in range finding study. Table 14 lists the runs parameters and Table 15 lists the results from the runs. The results indicate that the step recovery across the Protein A unit operation will increase with increasing column load and decreasing the elution pH, as what is commonly seen in a monoclonal antibody Protein A capture process. The center point condition gives the acceptable 70% step recovery. However, the results demonstrated that operation at high column loading and low elution pH conditions results in an increase in the level of aggregate in FNVIP samples. NVIP samples in this study were filtered only through 0.22 μm.

TABLE 14
D.O.E Parameters- Elution pH and Column Load

| | Range | |
|---|---|---|
| Parameter | Evaluated | Column ID/Volume (ml) |
| Column Load | 8.3-19.7 g/L | Omnifit ™ |
| Elution pH | 3.2-3.8 | 0.66 cm × 16 cm |
| | | ID/5.5 ml |

TABLE 15
D.O.E Results- Elution pH and Column Load

| Run | Elution | Column Load | Yield | High Molecular Weight |
|---|---|---|---|---|
| # | pH | (g/L) | (%) | (%) |
| HCCF | N/A | N/A | N/A | N/A |
| 1 | 3.7 | 18 | 56.5 | 0.4 |
| 2 | 3.5 | 14 | 70.0 | 3.01 |
| 3 | 3.5 | 14 | 71.2 | 2.91 |
| 4 | 3.3 | 10 | 81.7 | 1.96 |
| 5 | 3.7 | 10 | 38.0 | 1.06 |
| 6 | 3.3 | 18 | 75.5 | 2.93 |
| 7 | 3.5 | 8.3 | 58.5 | 0.6 |
| 8 | 3.2 | 14 | 73.5 | 2.48 |
| 9 | 3.5 | 14 | 69.6 | 2.90 |
| 10 | 3.5 | 19.7 | 72.5 | 1.97 |
| 11 | 3.5 | 14 | 69.6 | 3.22 |
| 12 | 3.8 | 14 | 30.1 | 1.10 |

### 3.1.7 Conclusion

The Protein A affinity chromatography, low pH viral inactivation, and filtration of neutralized viral inactivated product were evaluated for the purification of ISVD. Overall, due to the different structure of MSD-21, which lacks an Fc domain, compared to the structure of an antibody, which includes an Fc domain, modifications in the Protein A chromatography process for purifying MSD-21 were required to enable a Protein A chromatography process suitable for MSD-21 purification.

Table 16 lists representative operating parameters for MSD-21 Protein A purification. Based on the results shown herein, TOYOPEARL AF-rProtein A HC-650F was selected as the Protein A affinity resin with the highest binding capacity at 10% breakthrough (18 g/L) and which also gave the most MSD-21 aggregate removal among the other screened Protein A resins. The platform elution buffer (20 mM Na Acetate, PH 3.5) was also chosen as the elution buffer for the Protein A process since it provided the lowest MSD-21 product aggregation among the several elution conditions investigated. Five CV of each of wash2 and wash3 steps were settled upon to ensure respectively maximal removal of non-specifically bound impurities and declining the UV signal to baseline before starting the elution phase. The Protein A elution pool volume for MSD-21 was also observed to be larger than that observed in the typical Protein A process for purifying antibodies, therefore a longer elution step (8 CV) with UV signal auto-zeroing at the beginning of the step is suggested.

A robustness study for Protein A chromatography parameters showed that operating at the range of 9-18 grams protein/liter resin column load with a column load target of 18 grams protein/liter resin and at elution pH of 3.5±0.1 provides good performance and product quality.

A robustness study was conducted for the low pH viral inactivation step showing robust and stable inactivated product at pH 3.5±0.1 range for duration of up to 3 hours. The viral inactivation parameters are listed in Table 17.

TABLE 16
Protein A Affinity Chromatography Processing Parameters

| | | Buffer | Flow Rate | | | | Flow |
|---|---|---|---|---|---|---|---|
| Step | Solution | PH | (cm/hr) | Column Volume | | | Direction |
| Storage Buffer | 10 mM Sodium | 6.5 ± 0.2 | 300 | 3 | | | Down |
| Removal | Phosphate | | | | | | |
| Sanitization | 0.1 M Sodium | >12 | 300 | 3 CV Up, 20 minutes hold, 2 CV Down | | | |
| | Hydroxide | | | | | | |
| Equilibration | 10 mM Sodium | 6.5 ± 0.2 | 300 | ≥5 | | | Down |
| | Phosphate | | | | | | |
| Load | HCCF | FIO | 300 | 9-18 grams protein/liter resin | | | Down |
| Wash 1 | 10 mM Sodium | 6.5 ± 0.2 | 300 | 3 | | | Down |
| | Phosphate | | | | | | |
| Wash 2 | 10 mM Sodium | 6.5 ± 0.2 | 300 | 5 | | | Down |
| | Phosphate/ | | | | | | |
| | 0.5 M NaCl | | | | | | |
| Wash 3 | 10 mM Sodium | 6.5 ± 0.2 | 300 | 5 | | | Down |
| | Phosphate | | | | | | |
| Elution | 20 mM Sodium | 3.5 ± 0.1 | 300 | Action | MSD-21 | Total | Down |
| | Acetate | | | (CV) | (A280 | Approximate | |
| | | | | | AU/cm) | CV‡ | |
| | | | | Peak Start | 0.25 | ≤8 | |
| | | | | ~1 | | | |
| | | | | Pool Size | N.A. | | |
| | | | | 3-5 | | | |
| | | | | Post Pool | 0.25 | | |
| | | | | 1-2 | | | |

| Strip | 100 mM | 2.9 ± 0.2 | 300 | 3 | Up |
|---|---|---|---|---|---|
| | Acetic Acid | | | | |
| Flush | WFI | N/A | 300 | 3 | Up |
| Sanitization | 0.1 M Sodium | >12 | 300 | 3 CV Up, 20 min hold, 2 CV Down | |
| | Hydroxide | | | | |
| Storage | 2% (v/v) Benzyl | 5.0 ± 0.2 | 300 | 3 | Down |
| | 0.2 M Sodium | | | | |
| | Acetate, | | | | |
| | pH 5.0 | | | | |

FIO: For information only
‡‡UV auto-zeroed at the start of the elution block

TABLE 17
Viral Inactivation Parameters

| Inactivation Parameters | Target |
|---|---|
| Inactivation pH | 3.5 ± 0.1 |
| Inactivation Solution | 1M Acetic Acid |
| Inactivation Target Hold Time (min) | 60-90 |
| Neutralization Parameters | Target |
| Neutralized Viral Inactivated Product pH | 4.2 ± 0.1 |
| Neutralizing Solution | 1M Tris |

### Example 2

Complex formation between the V_{HH} domains and either MabSelect SuRe™ Protein A ligand (Mab Select Protein A ligand) or TOYOPEARL AF-r Protein A HC-650F Protein A ligand (Tosoh Protein A ligand) was performed by using high-resolution NMR titrations. NMR-based titrations provide the ability to monitor the interactions between different biomolecules at atomic resolution. These experiments report on target engagement, profile solution behavior (interaction dispersity), and importantly, can deduce the binding surface utilized to form the interaction. High resolution titrations are performed by adding ligand (i.e. titrant) in a step-wise manner to a sample containing isotope labeled (NMR active) material (Michielssens et al., Angew. Chem. Int. Ed. Engl. 2014, 53 (39), 10367-10371). For each step during the titration, a dataset is collected in which a spectrum contains individual peaks and each peak corresponds spectral signatures for two covalently bonded atoms. In order to achieve residue level resolution, the assignment of individual peaks within a 2D dataset is necessary. For isotope labeled anti-PD-1 V_{HH} and anti-CTLA4 V_{HH}, atomic assignments were determined using conventional triple resonance approaches (Sattler & Griesinger Prog. Nucl. Magn. Reson. Spect. 1999, 34, 93-158). About 94% and 98% complete backbone assignment for the isotope-labeled anti-PD-1 V_{HH} and anti-CTLA4 V_{HH} was obtained, respectively. This high level of assignment permitted quantitation of spectral changes that corresponded to nearly all peaks within each 2D [¹H^{N}, ¹⁵N]-HSQC (Heteronuclear Single Quantum Coherence) spectroscopy.

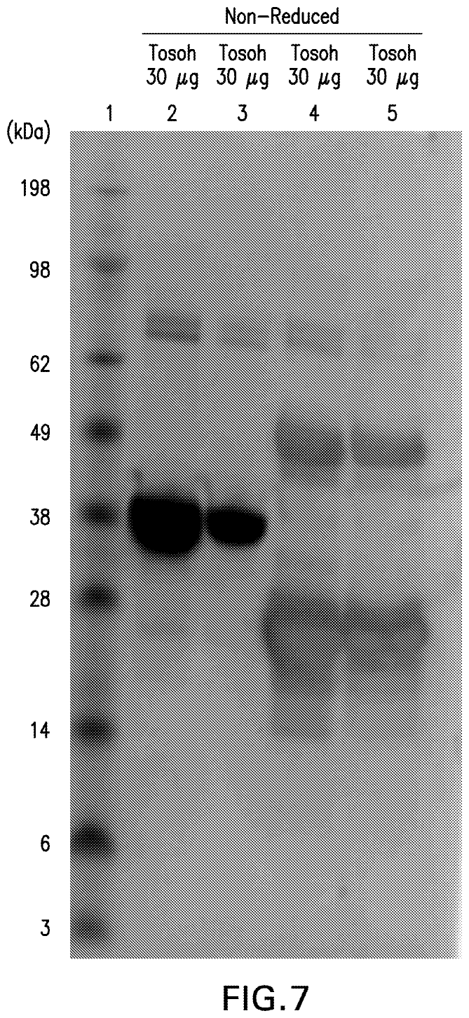

The addition of Protein A ligands to both V_{HH} domains caused differential peak broadening as a function of increased Protein A concentration. For both the Mab Select Protein A ligand and Tosoh Protein A ligand, at greater than or equal to 1 mg/mL, a dramatic transformation in the spectra is observed. At higher concentrations of Protein A ligand almost all observable peaks become very weak in intensity and are severely broadened. This indicates that both anti-PD-1 V_{HH} or anti-CTLA4 V_{HH} engage the Protein A ligands. The inability to be able to resolve all bound resonances (even under saturating conditions; V_{HH} domains are 1000 bound) from the complex indicates that the molecular weight has increased beyond the point of detection. These Protein A ligands are from commercial sources and details regarding their structural topology (e.g. number of potential binding sites, multivalent binding capacity, etc.) is unknown. The molecular weight of these ligands was estimated by SDS-PAGE analysis (FIG. 7). The major species from the Tosoh Protein A ligand (FIG. 7, lanes 2 and 3) run at approximately 38 kDa. It is important to note that Protein A from Staphylococcus aureus is a 34 kDa protein maintains five IgG binding sites and is very close to the molecular weight of the Tosoh Protein A ligand. Because the information around the number of potential binding sites is unavailable, it cannot be assumed the Tosoh Protein A ligand has the same multi-valency; however, its molecular weight indicates some similarity. A further complication is the observation that V_{HH} domains engage Protein A through a different structural surface as compared to full size monoclonal antibodies, which engage Protein A via the Fc domain. The unknown number of binding sites further complicates the ability to accurately measure the exact binding affinity between these molecules using calorimetric methods. The SDS-PAGE analysis of the Mab Select Protein A ligand reported two different dominant species of Protein A ligand under non-reducing denatured conditions, at approximately 50 kDa and 28 kDa (FIG. 7, lanes 4 and 5). If the V_{HH} domains form a monodisperse 1:1 complex in solution, the complex molecular weight is between 43 and 65 kDa. At these complex molecular weights and under the sample conditions and data acquisition parameters used here, the bound complexes are too large to be directly observed by 2D [¹H^{N}, ¹⁵N]-HSQC spectroscopy. This explains why there is severe peak broadening at Protein A ligands greater than 1 mg/mL. Importantly, by performing step-wise titrations the “rate” of peak disappearance, from samples with less than or equal to 1 mg/mL, can be used to quantitate the residues on the V_{HH} domain that are sensitive to the binding event.

### Quantitation of NMR-Based Titrations for the Elucidation of the V_{HH} Binding Surface to Protein a Ligands

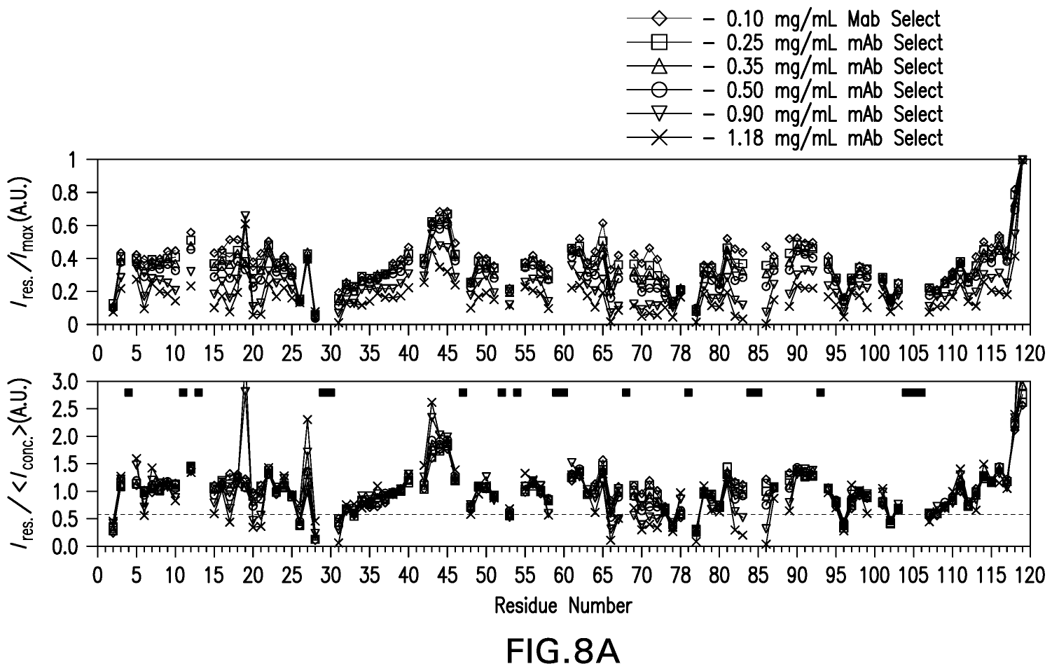

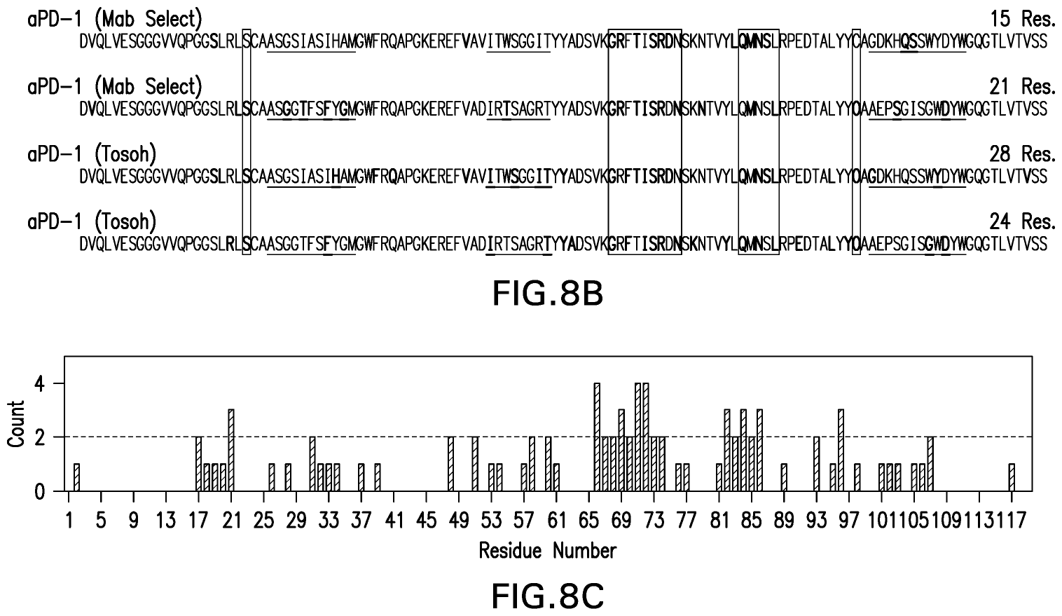

Within each dataset, the peak intensity was extracted for each resonance that could be assigned. Peak amplitudes were used as the bound peak resonance position could not be recovered and sufficiently quantitated. The broadening profile across all residues can be visualized by comparing the relative intensity for a given peak (Iᵣₑₛ) normalized with respect to the peak with the largest amplitude (Iₘₐₓ) within a spectrum. In the top panel of FIG. 8A, Iᵣₑₛ/Iₘₐₓ is plotted as a function of residue number for the aCTLA4:Mab Select Protein A ligand titration. As the concentration of Mab Select Protein A ligand was increased there is a uniform loss in average signal intensity. However, this data representation does not capture the “rate” at which some peaks may lose their intensity as compared to others. It is anticipated that for residues that are either directly engaged or for residues that are more strongly impacted to the binding event will have an increased loss in intensity as compared to other residues at a given titration point. The increased relative loss in intensity is because for residues strongly affected by binding causes induced rigidity which increases the relaxation rate of the NMR signal causing a more intense broadening for those amino acids. The lower panel of FIG. 8A highlights an analysis where residues most affected by binding can be discerned. The ratio of a given resonance (Iᵣₑₛ) as compared to the average intensity at a given concentration ( [symbol] I_{conc} [symbol] ) on of Protein A ligand more directly permits the identification of residues that are strongly affected by binding FIG. 8A; lower panel). For each titration, this analysis was performed and residues which had a Iᵣₑₛ/ [symbol] I_{conc} [symbol] less than [symbol] Iᵣₑₛ/ [symbol] I_{conc} [symbol] −σ_{I,conc} was selected to be impacted by the binding event (FIG. 8B). This approach reduces the bias from quantitation of peak amplitudes determined from NMR-based titration data.

[symbol]

Across all off the different titrations there were many residues, that span the primary sequence, which are impacted by binding to the different Protein A ligands (FIG. 8C). A distinction that emerges is the total number of residues which sense the binding event. The Tosoh Protein A ligand binding to both anti-PD-1 and anti-CTLA4 V_{HH} domains causes more residues to sense the binding event. The additional residues that contribute to this higher total flank CDR2 and is not captured by the Mab Select Protein A ligand. Note, that this does not necessarily imply that additional interactions are formed by Tosoh to both anti-PD-1 and anti-CTLA4 V_{HH} domains but identifies that Tosoh impacts V_{HH} molecules differently than Mab Select. This could stem to a partial sequestration of the CDR2 region upon engagement of Tosoh. This analysis also allows one to define a set of residues, common to both V_{HH} domains, that overlap across all titrations, and that are important on how Protein A ligands engage the V_{HH} scaffolds of the anti-PD-1 and anti-CTLA4 V_{HH} domains. Residues that form the common interaction site were given this designation if it was ranked as causing significant impact on binding in three or greater of the titrations (FIGS. 8A-8C). The identification of differences between anti-PD-1 and anti-CTLA4 V_{HH} domains with respect to the two Protein A ligands can be visualized by plotting residues that were qualified to strongly sense the binding event onto a molecular model of a V_{HH} domain.

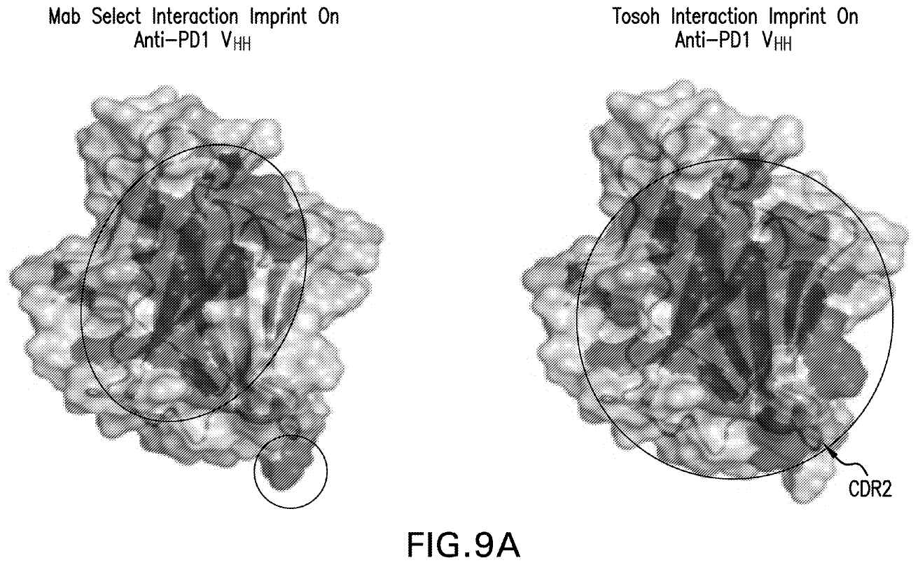

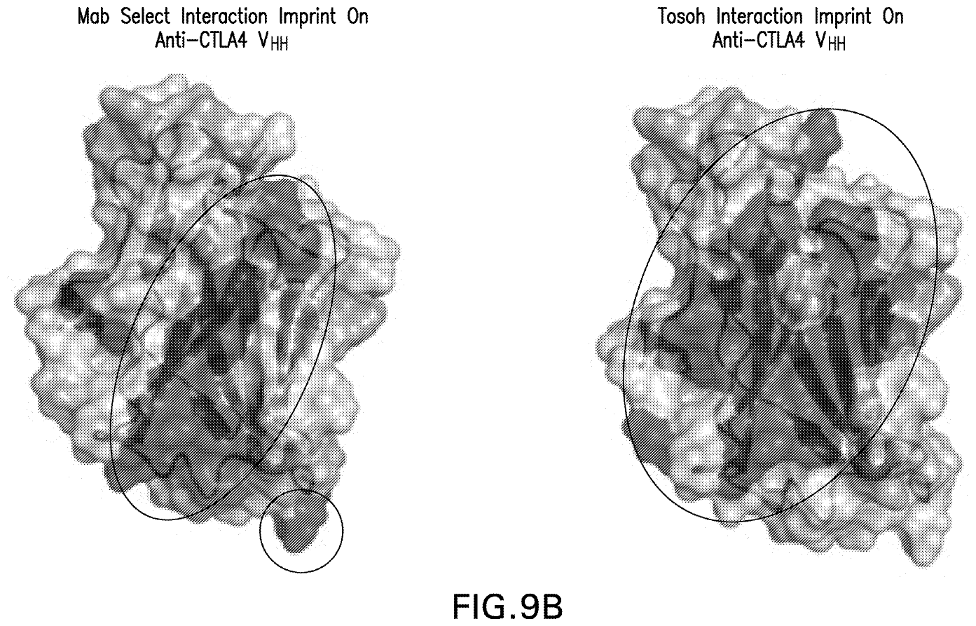

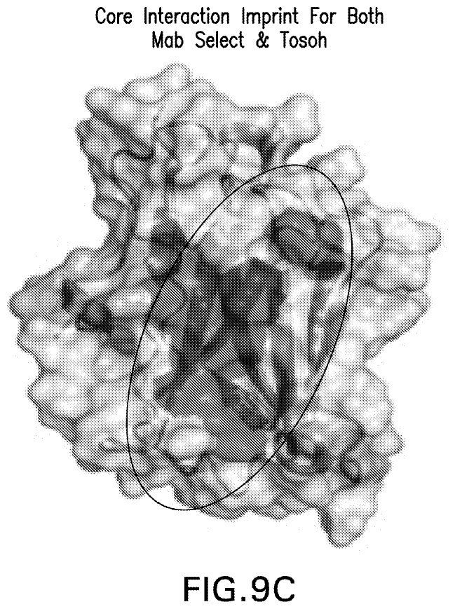

We have determined an atomic model of anti-PD-1 V_{HH} using an NMR-data restrained computer modeling approach (Shen et al., Proc. Natl. Acad. Sci. 2008, 105, 4685-4690). This model is well described by the experimental data used to restrain the atomic model and has a fold that is similar to other publicly disclosed atomic models (Graille et al., Proc. Natl. Acad. Sci. 2000, 97 5399-5404; Gerald et al., Amsphere JSR Life Sciences White Paper 2017, 1-12). This model was used to visualize the spatial localization of residues that were significantly impacted upon interaction with the different Protein A ligands (FIG. 8A-8B and FIG. 9A-9B). FIG. 9A-9B highlights the common and unique features across the different titrations. The surface representations of the anti-PD-1 and anti-CTLA4 V_{HH} domains in FIG. 9A-9B with darkened colors, correspond to residues that are impacted upon binding. In FIG. 9C, the residues from the common interaction surface bound by Mab Select and Tosoh are shown on the surface representation of the anti-PD-1 atomic model. The binding data imprinted on the structural representation of anti-PD-1 V_{HH} show that the core binding surface occurs within the anti-parallel f-strand scaffold between residues G66-S85 and is a patch that is located away from the CDR loops. The residues from the common interaction surface are highlighted and are shown on the bottom surface representation of the anti-PD-1 V_{HH} atomic model. Importantly, a larger contiguous surface patch is observed for the anti-PD-1 and anti-CTLA4 V_{HH} domains when engaged with the Tosoh Protein A ligand. This additional patch forms distal from the core interaction residues and involves residues that flank the CDR2 (residues 51-61) on both anti-PD-1 V_{HH} and anti-CTLA4 V_{HH}. This larger surface is not observed for both aPD-1 and aCTLA4 in complex with Mab Select Protein A ligand. This structural detail differentiates the behavior between the Tosoh Protein A ligand and Mab Select Protein A ligand interactions with V_{HH} domains.

### Materials and Methods

### Protein Biochemistry/NMR Spectroscopy

Uniformly labeled ¹³C and ¹⁵N labeled anti-PD-1 and anti-CTLA4 V_{HH} domains was overexpressed in Pichia pastoris and secreted to the medium after overexpression. Both protein reagents were purified using conventional column-based chromatography and subsequently flash frozen in a PBS buffer and stored at −80° C. Prior to any NMR experiments, all concentrated samples were thawed and then dialyzed into a buffer of 10 mM sodium phosphate at pH 6.6. Protein A ligands were kept in their formulated conditions and applied to dialyzed samples of V_{HH} domains. Note, Protein A stocks were kept at a higher concentration and diluted into each titration point sample. This was done to ensure that Protein A ligands were preserved in their recommended storage conditions prior to NMR-based titration. The V_{HH} domains that were used consisted of an anti-PD-1 V_{HH} and an anti-CTLA4 V_{HH}. For all multi-point titrations, the anti-PD-1 and anti-CTLA4 V_{HH} domains were kept at a constant concentration of 1.4 mg/mL (approximately 100 μM for both anti-PD-1 and anti-CTLA4 V_{HH} domains). Prior to collection of the titration datasets with Protein A ligands, both anti-PD-1 and anti-CTLA4 V_{HH} domains had their backbone assigned using a series of triple resonance experiments and a 3D-¹⁵N resolved NOESY (Nuclear Overhauser Effect Spectroscopy)-HSQC spectroscopy (Sattler & Griesinger Prog. Nucl. Magn. Reson. Spect. 1999, 34, 93-158). The Protein A ligands used here were of unknown structural topologies and were MabSelect SuRe™ (GE Healthcare) and Protein A-R40 Standard (ProteNova). TOYOPEARL AF-r Protein A HC-650F comprises the Protein A-R40 ligand and is referred to herein as the Tosoh Protein A ligand. The MabSelect SuRe™ Protein A ligand is referred to herein as the Mab Select Protein A ligand. The reported stock concentrations of both the Tosoh and Mab Select Protein A ligands were 29.8 and 2 mg/mL, respectively. The Mab Select Protein A ligand was not further concentrated to mitigate any potential aggregation. During the titrations between five and six different concentration points. The concentration of Mab Select Protein A ligand was varied between 0.1 and 1.18 mg/mL and the concentration of the Tosoh Protein A ligand was varied between 0.1 and 4 mg/mL. A 1.7 mm workflow was used for the NMR-based titrations permitting individual samples to be prepared for each point within the titration. All experiments were collected on a spectrometer operating at a Larmor frequency of 600 MHz and at a temperature of 300 K. For each sample a 2D [¹H^{N}, ¹⁵N]-HSQC spectroscopy and 1D experiment was collected. For each 2D dataset a total of 80 (t_{1,max}=42.4 ms) and 512 (t_{2,max}=63.9 ms) complex points in the indirect and direct dimension, respectively with 48 transients per point collected.

### Data Analysis

All NMR data were processed using a shifted sine-bell for adopization, zero-filled to 4,096 points in both dimensions, and a base line correction was applied in the direct dimension. Peak amplitudes were extracted from reach dataset within a given titration. Peak amplitudes were used to calculate the ratio of a given resonance (Iᵣₑₛ) over the maximum intensity (Iₘₐₓ) at a given titration point. The removal of overall impact due to increased molecular tumbling as a function of increased bound population was controlled for by converting the peak amplitudes to Iᵣₑₛ/ [symbol] I_{conc} [symbol] . This is a form of normalization across different concentrations. Residues were selected to be have impact on binding if a residue's Iᵣₑₛ/ [symbol] I_{conc} [symbol] value is less than [symbol] Iᵣₑₛ/ [symbol] I_{conc} [symbol] −σ_{I,conc} where σ_{I,conc} is the standard deviation of peak amplitudes across all residues.

Table of Sequences

| SEQ | | |
|---|---|---|
| ID | | |
| NO: | Description | Sequence |
| 1 | Anti-PD1 ISVD | IHAMG |
| | CDR 1 | |
| 2 | Anti-PD1 ISVD | VITWSGGITYYADSVKG |
| | CDR 2 (variant 1) | |
| 3 | Anti-PD1 ISVD | VITVSGGITYYADSVKG |
| | CDR 2 (variant 2) | |
| 4 | Anti-PD1 ISVD | DKHQSSWYDY |
| | CDR 3 (variant 1) | |
| 5 | Anti-PD1 ISVD | DKHQSSFYDY |
| | CDR 3 (variant 2) | |
| 6 | Anti-LAG3 ISVD | DYVMG |
| | CDR 1 | |
| 7 | Anti-LAG3 ISVD | AISESGGRTH |
| | CDR 2 | |
| 8 | Anti-LAG3 ISVD | TLLWWTSEYAPIKANDYDY |
| | CDR 3 | |
| 9 | Anti-CTLA4 ISVD | GGTFSFYGMG |
| | 011F01 CDR 1 | |
| 10 | Anti-CTLA4 ISVD | DIRTSAGRTY |
| | CDR 2 | |
| 11 | Anti-CTLA4 ISVD | EMSGISGWDY |
| | CDR 3 (variant 1) | |
| 12 | Anti-CTLA4 ISVD | EPSGISGWDY |
| | CDR 3 (variant 2) | |
| 13 | Anti-HSA ISVD | SFGMS |
| | CDR 1 | |
| 14 | Anti-HSA ISVD | SISGSGSDTL |
| | CDR 2 | |
| 15 | Anti-HSA ISVD | GGSLSR |
| | CDR 3 | |
| 16 | Anti-PD1 | DVQLVESGGGVVQPGGSLRL |
| | VHH | SCAASGSIASIHAMGWFRQA |
| | (ISVD) | PGKEREFVAVITWSGGITYY |
| | | ADSVKGRFTISRDNSKNTVY |
| | | LQMNSLRPEDTALYYCAGDK |
| | | HQSSWYDYWGQGTLVTVSS |
| 17 | Anti-PDl VHH | EVQLVESGGGVVQPGGSLRL |
| | (ISVD) DIE | SCAASGSIASIHAMGWFRQA |
| | variant | PGKEREFVAVITWSGGITYY |
| | | ADSVKGRFTISRDNSKNTVY |
| | | LQMNSLRPEDTALYYCAGDK |
| | | HQSSWYDYWGQGTLVTVSS |
| 18 | Anti-LAG3 VHH | EVQLVESGGGVVQPGGSLRL |
| | (ISVD) | SCAASGRTFSDYVMGWFRQA |
| | | PGKEREFVAAISESGGRTHY |
| | | ADSVKGRFTISRDNSKNTLY |
| | | LQMNSLRPEDTALYYCATTL |
| | | LWWTSEYAPIKANDYDYWGQ |
| | | GTLVTVSS |
| 19 | Anti-LAG3 VHH | DVQLVESGGGVVQPGGSLRL |
| | (ISVD) | SCAASGRTFSDYVMGWFRQA |
| | E1D variant | PGKEREFVAAISESGGRTHY |
| | | ADSVKGRFTISRDNSKNTLY |
| | | LQMNSLRPEDTALYYCATTL |
| | | LWWTSEYAPIKANDYDYWGQ |
| | | GTLVTVSS |
| 20 | Anti-HSA | EVQLVESGGGWQPGNSLRLS |
| | VHH | CAASGFTFSSFGMSWVRQAP |
| | (ISVD) | GKGLEWVSSISGSGSDTLYA |
| | | DSVKGRFTISRDNAKTTLYL |
| | | QMNSLRPEDTALYYCTIGGS |
| | | LSRSSQGTLVTVSS |
| 21 | Anti-HSA | DVQLVESGGGVVQPGNSLRL |
| | VHH | SCAASGFTFSSFGMSWVRQA |
| | (ISVD) | PGKGLEWVSSISGSGSDTLY |
| | E1D variant | ADSVKGRFTISRDNAKTTLY |
| | | LQMNSLRPEDTALYYCTIGG |
| | | SLSRSSQGTLVTVSS |
| 22 | Anti-CTLA4 | DVQLVESGGGVVQPGGSLRL |
| | VHH | SCAASGGTFSFYGMGWFRQA |
| | (ISVD) | PGKEREFVADIRTSAGRTYY |
| | | ADSVKGRFTISRDNSKNTVY |
| | | LQMNSLRPEDTALYYCAAEP |
| | | SGISGWDYWGQGTLVTVSS |
| 23 | Anti-CTLA4 | EVQLVESGGGVVQPGGSLRL |
| | VHH | SCAASGGTFSFYGMGWFRQA |
| | (ISVD) | PGKEREFVADIRTSAGRTYY |
| | DIE variant | ADSVKGRFTISRDNSKNTVY |
| | | LQMNSLRPEDTALYYCAAEP |
| | | SGISGWDYWGQGTLVTVSS |
| 24 | MSD-21 | DVQLVESGGGVVQPGGSLRL |
| | polypeptide | SCAASGSIASIHAMGWFRQA |
| | (PD1)- | PGKEREFVAVITWSGGITYY |
| | (LAG3)-(HSA) | ADSVKGRFTISRDNSKNTVY |
| | | LQMNSLRPEDTALYYCAGDK |
| | | HQSSWYDYWGQGTLVTVSSG |
| | | GGGSGGGGSGGGGSGGGGSG |
| | | GGGSGGGGSGGGGSEVQLVE |
| | | SGGGVVQPGGSLRLSCAASG |
| | | RTFSDYVMGWFRQAPGKERE |
| | | FVAAISESGGRTHYADSVKG |
| | | RFTISRDNSKNTLYLQMNSL |
| | | RPEDTALYYCATTLLWWTSE |
| | | YAPIKANDYDYWGQGTLVTV |
| | | SSGGGGSGGGGSGGGGSGGG |
| | | GSGGGGSGGGGSGGGGSEVQ |
| | | LVESGGGVVQPGNSLRLSCA |
| | | ASGFTFSSFGMSWVRQAPGK |
| | | GLEWVSSISGSGSDTLYADS |
| | | VKGRFTISRDNAKTTLYLQM |
| | | NSLRPEDTALYYCTIGGSLS |
| | | RSSQGTLVTVSSA |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1: A process for separating antigen-binding polypeptide (ABP) monomers from aggregates of the ABP monomers in a harvested cell culture fluid (HCCF), comprising:
(a) providing a HCCF from a culture of recombinant cells expressing ABP monomers, wherein the HCCF comprises a mixture of the ABP monomers, aggregates of the ABP monomers, and other protein, and a chromatography column comprising a Protein A resin selected from the group consisting of TOYOPEARL AF-rProtein A HC-650F resin and AMSPHERE A3 resin, equilibrated in an equilibration solution at a slightly acidic pH or neutral pH;
(b) applying the HCCF to the chromatography column;
(c) washing the chromatography column with at least one wash solution at a slightly acidic pH or neutral pH; and
(d) eluting the ABP monomers from the chromatography column with an elution solution at about pH 3.5 to obtain an eluent comprising the ABP monomer, wherein less than about 5% of the ABP monomers in the eluant are aggregates of the monomers as determined by ultra-performance size exclusion chromatography and wherein an ABP monomer comprises one or more immunoglobulin single variable domains (ISVDs).

2: The process of claim 1, wherein in step (d) less than about 2% of the ABP monomers in the eluant are aggregates of the monomers as determined by ultra-performance size exclusion chromatography.

3: The process of claim 1, wherein the HCCF is applied to the column at a continuous flow rate until the amount of total protein applied to the column reaches about 10% breakthrough or the amount of total protein applied to the column is about 9 to 18 grams protein/liter resin.

4: The process of claim 1, wherein the total protein concentration of the HCCF comprises about 1.0 grams/liter to about 1.5 grams/liter of protein.

5: The process of claim 1, wherein the chromatography column is equilibrated using an equilibration solution comprising 10 mM sodium phosphate.

6: The process of claim 1, wherein the at least one wash solution comprises 10 mM sodium phosphate.

7: The process of claim 1, wherein column is washed with a first wash solution comprising 10 mM sodium phosphate and a second wash solution comprising 10 mM sodium phosphate and 500 mM sodium chloride.

8: The process of claim 7, wherein the column is washed in the following order: the first wash solution for about three column volumes, the second wash solution for about five column volumes, and the first wash solution for about three column volumes.

9: The process of claim 1, wherein the elution solution comprises of 20 mM sodium acetate.

10: The process of claim 1, wherein the slightly acidic pH is a pH that is greater than about pH 6.0 and less than pH 7.0 and a neutral pH is a pH of 7.0 to about 7.5.

11: The process of claim 1, wherein each of the ISVDs comprises a humanized Camelid variable heavy domain (VHH) and wherein the VHH comprises an arginine residue at position 19 and an asparagine residue at position 82a wherein the position numbers are according to Kabat.

12: The process of claim 1, wherein the at least one ISVD comprises an amino acid sequence that binds programmed death receptor 1 (PD-1).

13: The process of claim 1, wherein the ABP monomer comprises at least two ISVDs wherein one ISVD has an amino acid sequence that binds programmed death receptor 1 (PD-1) and the other ISVDs have amino acid sequences that bind another antigens.

14-23. (canceled)

24: A composition comprising antigen-binding polypeptide (ABP) monomers and a pharmaceutically acceptable carrier, wherein less than about 5% of the ABP monomers in the composition are aggregates of the monomers as determined by ultra-performance size exclusion chromatography, wherein the composition has a pH of 3.5 pH units, and wherein an ABP monomer comprises one or more immunoglobulin single variable domains (ISVDs) (Original):

25: The composition of claim 24, wherein in less than about 2% of the ABP monomers in the composition are aggregates of the monomers as determined by ultra-performance size exclusion chromatography.

26: The composition of claim 24, wherein each of the ISVDs comprises a humanized Camelid variable heavy domain (VHH).

27: The composition of claim 26, wherein each of the VHH comprises an arginine residue at position 19 and an asparagine at position 82a wherein the position number is according to Kabat.

28: The composition of claim 24, wherein at least one ISVD comprises an amino acid sequence that binds programmed death receptor 1 (PD-1).

29: The composition of claim 24, wherein the ABP monomers comprise at least two ISVDs wherein one ISVD has an amino acid sequence that binds programmed death receptor 1 (PD-1) and the other ISVDs have amino acid sequences that bind another antigens.

30-39. (canceled)
